(19) United States
(12) Patent Application Publication
(10) Pub. No.: US 2011/0275695 A1
(43) Pub. Date: Nov. 10, 2011

(54) CRYSTALLINE FORMS OF ZOTEPINE HYDROCHLORIDE

(75) Inventors: Isabel Kalofonos, Cambridge, MA (US); Dimitris Kalofonos, Cambridge, MA (US); William Martin-Doyle, Newton, MA (US); Jason Hanko, West Lafeyette, IN (US); Eric J. Hagen, Lafayette, IN (US)

(73) Assignee: BIONEVIA PHARMACEUTICALS, INC., Cambridge, MA (US)

(21) Appl. No.: 12/997,499

(22) PCT Filed: Jun. 11, 2009
(86) PCT No.: PCT/US2009/047060

§ 371 Date: Jul. 26, 2011

Related U.S. Application Data
(60) Provisional application No. 61/061,253, filed on Jun. 13, 2008.

(51) Int. Cl. A61K 31/38; A61P 25/00; A61P 25/22; A61P 25/14; A61P 19/06; C07D 337/14; A61P 25/18

(52) U.S. Cl. 514/431; 549/12

(57) ABSTRACT

The invention relates to crystalline forms of zotepine hydrochloride, including the crystalline hydrochloride salt of zotepine and two cocrystals of zotepine hydrochloride with benzoic acid. The preparation and characterization of these crystalline forms of zotepine hydrochloride is described. The invention also relates to the therapeutic use of the crystalline forms of zotepine hydrochloride to treat central nervous system disorders and to pharmaceutical compositions containing them.

### CROSS-REFERENCE TO RELATED APPLICATIONS

This application claims priority under 35 U.S.C. §119 to U.S. Provisional Application 61/061,253, filed Jun. 13, 2008, which is incorporated herein by reference.

### FIELD OF THE INVENTION

The invention relates to crystalline forms of zotepine hydrochloride, including the crystalline hydrochloride salt of zotepine and two cocrystal forms of zotepine hydrochloride with benzoic acid. The invention also relates to their therapeutic use to treat central nervous system disorders and to pharmaceutical compositions containing them.

### BACKGROUND OF THE INVENTION

Zotepine, 2-[(8-chlorodibenzo[b,f]thiepin-10-yl)oxy]-N,N-dimethylethylamine, (shown below) is a known active pharmaceutical ingredient (API) having beneficial central nervous system activity and is useful in treating central nervous system conditions.

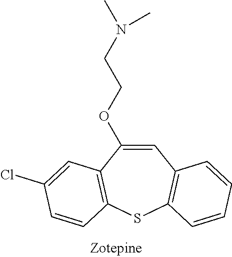

For example, zotepine is therapeutically effective in the treatment of schizophrenia and psychosis. Zotepine also has positive indications for the treatment of cognitive symptoms of schizophrenia or psychosis, negative symptoms of schizophrenia or psychosis, bipolar disorder, Huntington's Disease, behavioral and psychological symptoms of dementia, pain, gout, depression, and anxiety disorders. The preparation and pharmacologic activity of zotepine are described in U.S. Pat. No. 3,704,245 and in British Patent Specification 1,247,067. Therapeutic activity in various conditions has been demonstrated in the clinical literature, including but not limited to Kasper, S. et al, Int Clin Psychopharmacol 2001 16 163-168; Cooper, S. J. et al, Psychopharmacology (Berlin) 2000 150 237-243; Meyer-Lindenberg, A. et al, Pharmacopsychiatry 1997 30 35-42; Petit, M. et al, Psychopharmacol Bull. 1996 32 81-87; Hashimoto, K. et al, Schizophr Research 2006 87 332-333; Amann, B. et al, Bipolar Disord. 2005 7 471-476; Harada, T. et al, Clin Ther. 1986 8 406-414; Bonelli, R. M. et al, Hum Psychopharmacol. 2003 18 227-229; and Rainer, M. K. et al, CNS Drugs 2004 18 49-55; and in the patent literature, including U.S. Pat. No. 4,443,469; U.S. Pat. No. 6,444,665; U.S. Pat. No. 6,936,601; and WO9723477.

Although therapeutic efficacy is the primary concern for a therapeutic agent, like zotepine, the salt and solid state form (i.e., the crystalline or amorphous form) of a drug candidate can be critical to its pharmacological properties and to its development as a viable API. For example, each salt or each crystalline form of a drug candidate can have different solid state (physical and chemical) properties. The differences in physical properties exhibited by a novel solid form of an active pharmaceutical ingredient (API), (such as a cocrystal, salt, or polymorph of the original compound), affect pharmaceutical parameters such as storage stability, compressibility and density (important in formulation and product manufacturing), and solubility and dissolution rates (important factors in determining bioavailability). Because these practical physical properties are influenced by the solid state form of the API, they can significantly impact the selection of a compound as an API, the ultimate pharmaceutical dosage form, the optimization of manufacturing processes, and absorption in the body. Moreover, finding the most adequate form for further drug development can reduce the time and the cost of that development.

Obtaining pure crystalline forms, then, is extremely useful in drug development. It permits better characterization of the drug candidate's chemical and physical properties. Crystalline forms often have better chemical and physical properties than the amorphous state. The crystalline form may possess more favorable pharmacology than the amorphous form or be easier to process. It may also have better storage stability.

One such physical property, which can affect processability, is the flowability of the solid, before and after milling, Flowability affects the ease with which the material is handled during processing into a pharmaceutical composition. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a pharmaceutical compound is its dissolution rate in aqueous fluid. The rate of dissolution of an active ingredient in a patient's stomach fluid may have therapeutic consequences since it impacts the rate at which an orally administered active ingredient may reach the patient's bloodstream.

Another important solid state property of a pharmaceutical compound is its thermal behavior, including its melting point. The melting point of the solid form of a drug must be high enough to avoid melting or plastic deformation during standard processing operations, as well as concretion of the drug by plastic deformation on storage (Gould, P. L. Int. J. Pharmaceutics 1986 33 201-217). Normally a solid form should melt above about 100° C. to be considered optimum for development. For example, melting point categories used by one pharmaceutical company are, in order of preference, +(mp>120° C.), 0 (mp 80-120° C.), and −(mp<80° C.) (Balbach, S.; Korn, C. Int. J. Pharmaceutics 2004 275 1-12).

It is also possible to achieve desired properties of a particular API by forming a cocrystal of the API itself or of a salt of the API. Cocrystals are crystals that contain two or more non-identical molecules. Examples of cocrystals may be found in the Cambridge Structural Database. Examples of cocrystals may also be found at Etter, M. C., and Adsmond, D. A., J. Chem. Soc., Chem. Commun. 1990 589-591; Etter, M. C., MacDonald, J. C., and Bernstein, J., Acta Crystallogr., Sect. B, Struct. Sci. 1990 B46 256-262; and Etter, M. C., Urbańczyk-Lipkowska, Z., Zia-Ebrahimi, M., and Panunto, T. W., J. Am. Chem. Soc. 1990 112 8415-8426, which are incorporated herein by reference in their entireties. The following articles are also incorporated herein by reference in their entireties: Görbotz C. H., and Hersleth, H. P. Acta Cryst. 2000 B56 625-534; and Senthil Kumar, V. S., Nangia, A., Katz, A. K., and Carrell, H. L., Crystal Growth & Design, 2002 2 313-318.

By cocrystallizing an API or a salt of an API with a co-former (the other component of the cocrystal), one creates a new solid state form of the API which has unique properties compared with existing solid forms of the API or its salt. For example, a cocrystal may have different dissolution and solubility properties than the active agent itself or its salt. Cocrystals containing APIs can be used to deliver APIs therapeutically. New drug formulations comprising cocrystals of APIs with pharmaceutically acceptable co-formers may have superior properties over existing drug formulations.

A crystalline form of a compound, a crystalline salt of the compound or a cocrystal containing the compound or its salt form generally possesses distinct crystallographic and spectroscopic properties when compared to other crystalline forms having the same chemical composition. Crystallographic and spectroscopic properties of the particular form are typically measured by X-ray powder diffraction (XRPD), single crystal X-ray crystallography, solid state NMR spectroscopy, e.g. ¹³C CP/MAS NMR, or Raman spectrometry, among other techniques. The particular crystalline form of a compound, of its salt, or of a cocrystal often also exhibit distinct thermal behavior. Thermal behavior is measured in the laboratory by such techniques as capillary melting point, thermogravimetric analysis (TGA) and differential scanning calorimetry (DSC).

As mentioned above, U.S. Pat. No. 3,704,245 describes the synthesis and basic activities of a family of compounds including zotepine. The zotepine free base form is reported to be relatively insoluble in water, with a low dissolution rate. The low aqueous solubility and dissolution rate of the zotepine free base negatively impact the bioavailability of pharmaceutical formulations containing the zotepine free base, which has been measured at 7-13%.

Zotepine free base melts at about 90-91° C. (Merck Index, 13^{th} edition, 2001). Since the melting point of a solid form of a drug must be high enough to avoid melting or plastic deformation during standard processing operations, as well as concretion of the drug by plastic deformation on storage, higher melting points than this are normally preferred.

Accordingly, there is a need in the art to both increase the bioavailability of zotepine and to improve upon the thermal behavior of the free base. This invention answers those needs by providing crystalline forms of zotepine hydrochloride with improved properties, e.g, manufacturing properties and/or pharmacological properties. The invention also relates to processes of preparing those crystalline forms of zotepine hydrochloride, pharmaceutical compositions containing them, and their use to treat central nervous system conditions.

### SUMMARY OF THE INVENTION

The invention relates to crystalline forms of zotepine hydrochloride, including the crystalline hydrochloride salt of zotepine and two cocrystal forms of zotepine hydrochloride salt and benzoic acid. These novel forms exhibit improved thermal behavior, aqueous solubility, and dissolution rates in comparison to the previously known zotepine free base.

### BRIEF DESCRIPTION OF THE FIGURES

FIG. 1 depicts a representative XRPD pattern of crystalline benzoic acid.

FIG. 2 depicts a representative XRPD pattern of crystalline zotepine free base.

FIG. 3 depicts representative DSC/TGA analyses of crystalline zotepine free base.

FIG. 4 depicts the proton NMR spectrum of zotepine free base.

FIG. 5 depicts three UV absorbance vs. time curves from the intrinsic dissolution experiment for crystalline zotepine free base in water at 25° C.

FIG. 6 depicts a representative XRPD pattern of crystalline zotepine hydrochloride.

FIG. 7 depicts the DSC/TGA analyses of crystalline zotepine hydrochloride.

FIG. 8 depicts the proton NMR spectrum of zotepine hydrochloride.

FIG. 9 depicts the Raman spectrum of crystalline zotepine hydrochloride.

FIG. 10 depicts the intrinsic dissolution curves for crystalline zotepine hydrochloride in water at 25° C.

FIG. 11 depicts the XRPD pattern of the 1:1 zotepine hydrochloride benzoic acid cocrystal.

FIG. 12 depicts the proton NMR spectrum of the 1:1 zotepine hydrochloride benzoic acid cocrystal.

FIG. 13 depicts the DSC/TGA analyses of the 1:1 zotepine hydrochloride benzoic acid cocrystal.

FIG. 14 depicts the FT-Raman spectrum of the 1:1 zotepine hydrochloride benzoic acid cocrystal.

FIG. 15 depicts the intrinsic dissolution curves for the 1:1 zotepine hydrochloride benzoic acid cocrystal in water at 25° C.

FIG. 16 is an ORTEP drawing of 1:1 zotepine hydrochloride benzoic acid cocrystal. Atoms are represented by 50% probability anisotropic thermal ellipsoids.

FIG. 17 is the packing diagram of 1:1 zotepine hydrochloride benzoic acid cocrystal viewed down the crystallographic c axis.

FIG. 18 shows the hydrogen bonding scheme for 1:1 zotepine hydrochloride benzoic acid cocrystal. Hydrogen bonds are represented as dashed lines.

FIG. 19 compares the experimental and calculated XRPD patterns of 1:1 zotepine hydrochloride benzoic acid cocrystal.

FIG. 20 depicts the XRPD pattern of the 2:1 zotepine hydrochloride benzoic acid cocrystal.

FIG. 21 depicts the DSC/TGA analyses of the 2:1 zotepine hydrochloride benzoic acid cocrystal.

FIG. 22 depicts the proton NMR spectrum of 2:1 zotepine hydrochloride benzoic acid cocrystal.

FIG. 23 depicts the FT-Raman spectrum of the 2:1 zotepine hydrochloride benzoic acid cocrystal.

FIG. 24 depicts the intrinsic dissolution curves for the 2:1 zotepine hydrochloride benzoic acid cocrystal in water at 25° C.

FIG. 25 shows the intrinsic dissolution comparison between the zotepine hydrochloride salt, 2:1 zotepine hydrochloride benzoic acid cocrystal, and 1:1 zotepine hydrochloride benzoic acid cocrystal in water at 25° C. (top to bottom).

FIG. 26 compares the XRPD patterns of crystalline zotepine free base, crystalline zotepine hydrochloride salt, 1:1 zotepine hydrochloride benzoic acid cocrystal, and benzoic acid (top to bottom).

FIG. 27 compares the XRPD patterns of crystalline zotepine free base, crystalline zotepine hydrochloride salt, 2:1 zotepine hydrochloride benzoic acid cocrystal, and benzoic acid (top to bottom).

FIG. 28 compares the XRPD patterns for the 1:1 zotepine hydrochloride benzoic acid cocrystal and the 2:1 zotepine hydrochloride benzoic acid cocrystal (top to bottom).

### DETAILED DESCRIPTION OF THE INVENTION

The invention relates to crystalline forms of zotepine hydrochloride. Specifically, the inventive crystalline forms include the crystalline hydrochloride salt of zotepine, crystalline zotepine hydrochloride, and two cocrystal forms of zotepine hydrochloride salt with benzoic acid, a 1:1 zotepine hydrochloride benzoic acid cocrystal and a 2:1 zotepine hydrochloride benzoic acid crystal. The crystalline forms of the invention exhibit improved properties, including improved thermal behavior, aqueous solubility, and dissolution rates, in comparison to the known zotepine free base. The crystalline zotepine hydrochloride has significantly higher aqueous solubility and dissolution rate compared to the known zotepine free base. The two cocrystal forms of zotepine hydrochloride with benzoic acid possess aqueous solubilities and dissolution rates intermediate between zotepine free base and zotepine hydrochloride salt. Thus, the cocrystals of the invention ensure an appropriate range of options for speed of release between this fast-dissolving crystalline hydrochloride salt and the slower-dissolving zotepine free base. The crystalline forms of the invention also exhibit higher melting points in comparison to zotepine free base. The preparation of the crystalline forms of the invention, crystalline zotepine hydrochloride, the 1:1 zotepine hydrochloride benzoic acid cocrystal, and the 2:1 zotepine hydrochloride benzoic acid cocrystal are described below in the examples.

### Crystalline Zotepine Hydrochloride

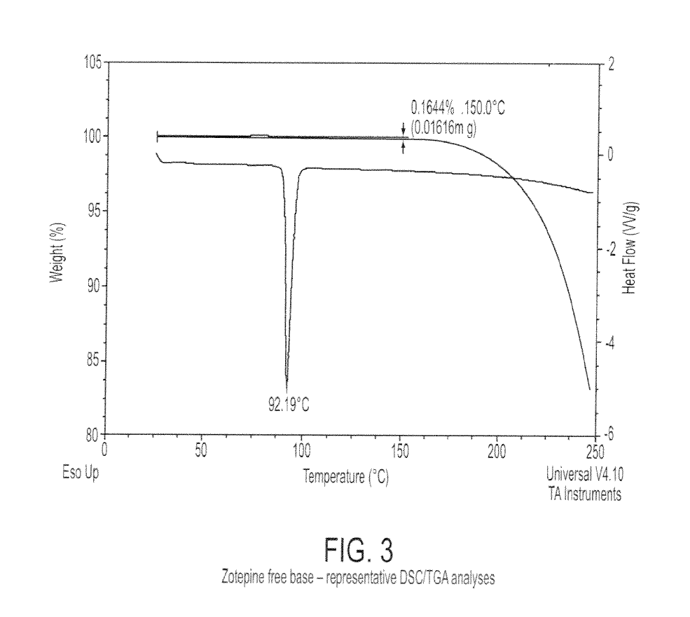

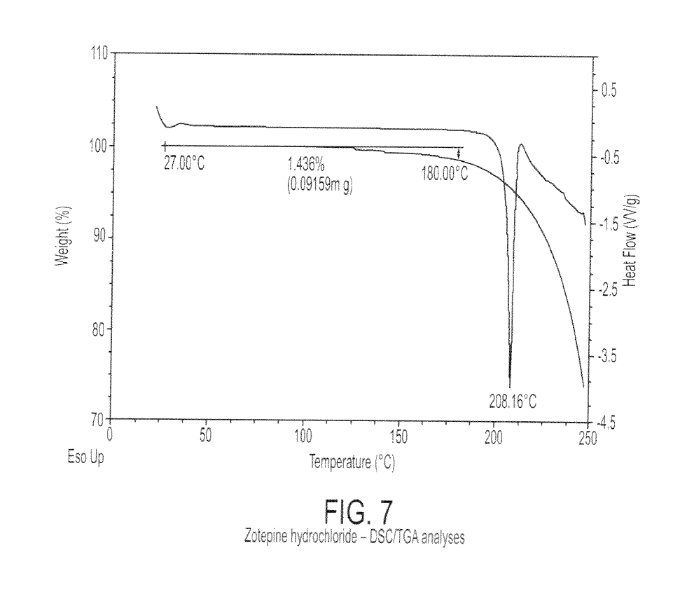

Zotepine hydrochloride was obtained in a crystalline solid form which is characterized by a unique x-ray powder diffraction pattern, a unique melting point, and a unique Raman spectrum. Crystalline zotepine hydrochloride was found to have improved thermal characteristics, aqueous solubility, and dissolution rate compared to zotepine free base. Zotepine free base melts at about 90-91° C. (Merck Index. 13^{th} edition, 2001), confirmed by the DSC trace in FIG. 3 which shows a sharp endotherm at about 92° C. Zotepine hydrochloride melts at about 208° C., as shown by the DSC trace in FIG. 7. Use of zotepine hydrochloride may avoid potential problems that could arise from plastic deformation of zotepine free base during storage and processing.

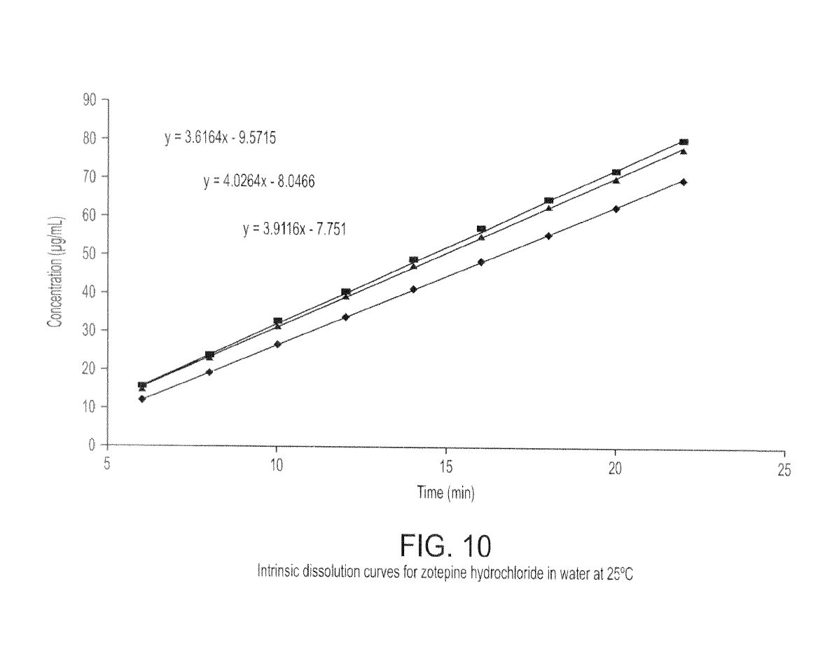

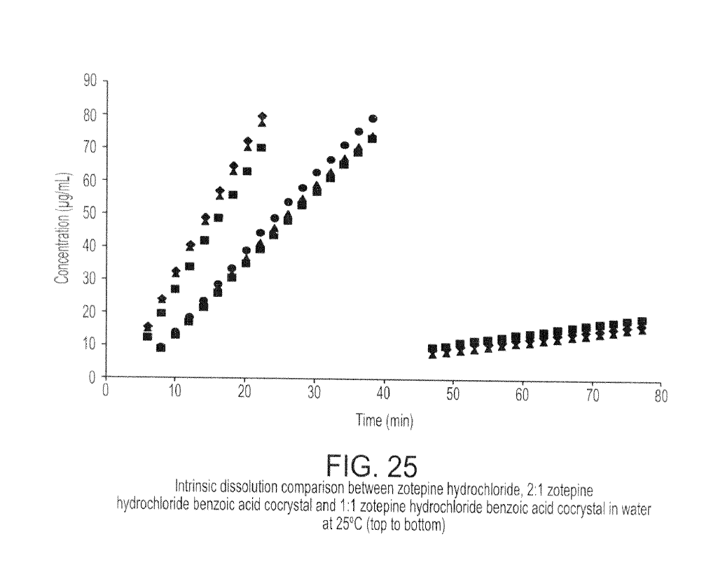

Crystalline zotepine hydrochloride was found to be considerably more rapidly dissolved in water compared to zotepine free base. The average dissolution rate of zotepine hydrochloride (three replicates) is about 3.9 [μg/mL]/min (as shown in FIGS. 10 and 25) compared to a rate very close to zero for zotepine free base. Zotepine free base is not plotted in units of concentration vs. time on FIG. 25, since the negligible recorded UV absorbance values for the free base fall below the minimum absorbance value measured for the Beer's Law Plot relationship generated from zotepine hydrochloride salt aqueous standards; the recorded absorbance values were also smaller than the inherent variability of the measurement. However, if zotepine free base concentration values were calculated, those values would track along the x axis. The very low dissolution rate of zotepine free base, and the negative effect of that rate on bioavailability, can be overcome by using crystalline zotepine hydrochloride.

Attempts to measure the equilibrium solubility of zotepine hydrochloride in water suggested that zotepine hydrochloride exhibits high surface activity. Addition of aliquots of solid zotepine hydrochloride to water resulted in the apparent dissolution of all aliquots, with the formation of a sudsy solution based on visual inspection. Additions were stopped at a concentration of >400 mg/mL, which is higher than expected based on the measured dissolution rate for zotepine hydrochloride. In some cases surface activity in a drug is disadvantageous from a formulation point of view (Perpesypkin, A.; Kwei, G.; Ellison, M.; Lynn, K.; Zhang, D.; Rhodes, T.; Remenar, J. Pharm. Res. 2005 22 1438 1444).

### 1:1 Zotepine Hydrochloride Benzoic Acid Cocrystal

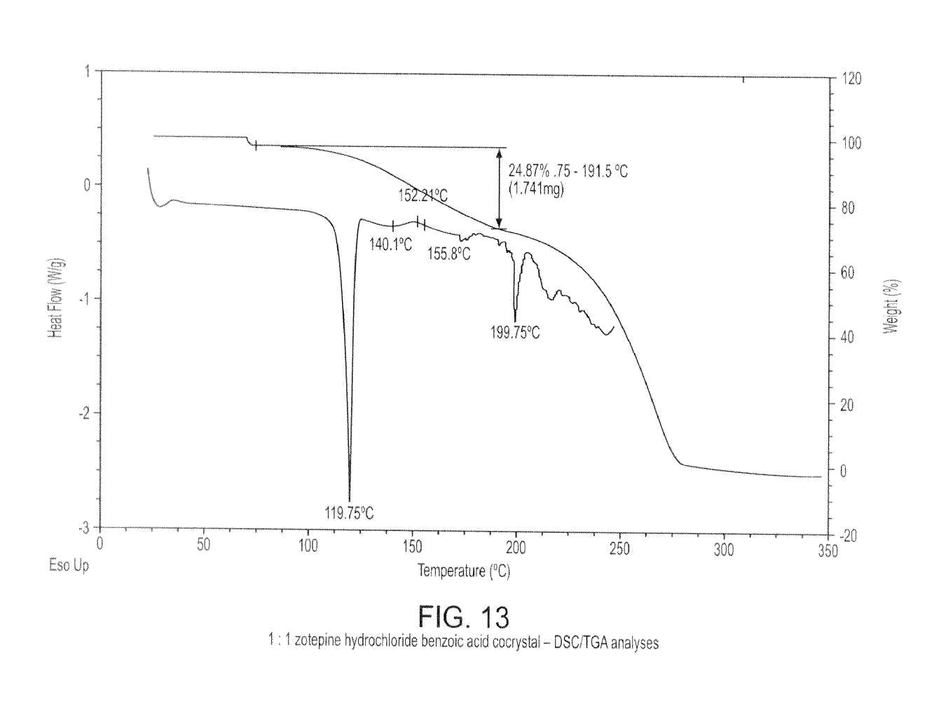

A 1:1 cocrystal of zotepine hydrochloride and benzoic acid was obtained in a crystalline solid form which is characterized by a unique x-ray powder diffraction pattern, a unique melting point, and a unique Raman spectrum. The crystal structure of the 1:1 zotepine hydrochloride benzoic acid cocrystal was determined by single-crystal x-ray diffraction analysis. The 1:1 zotepine hydrochloride benzoic acid cocrystal was found to have an acceptable melting point, about 119° C., as shown by the DSC trace in FIG. 13. Its dissolution rate was found to be intermediate between those of zotepine free base and zotepine hydrochloride, as shown in FIG. 25 (zotepine free base not plotted, as negligible recorded UV absorbance values fall below the minimum absorbance value measured for the Beer's Law Plot relationship generated from zotepine hydrochloride salt aqueous standards), which provides the ability to tune the dissolution rate of the drug to the desired level based on the therapeutic use and specific formulation desired. The equilibrium solubility of this 1:1 cocrystal in water was estimated to be 44 mg/mL by adding aliquots of solid 1:1 zotepine hydrochloride benzoic acid cocrystal to water until solids persisted, followed by removal of the solids and measurement of the concentration in solution.

### 2:1 Zotepine Hydrochloride Benzoic Acid Cocrystal

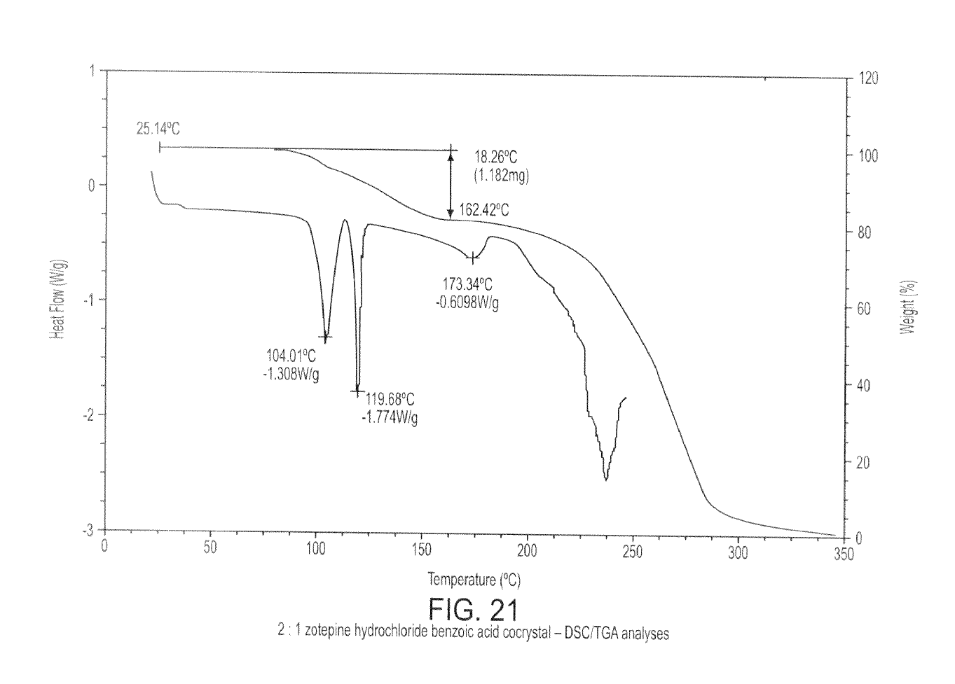

A 2:1 cocrystal of zotepine hydrochloride and benzoic acid was obtained in a crystalline solid form which is characterized by a unique x-ray powder diffraction pattern, a unique melting point, and a unique Raman spectrum. The 2:1 zotepine hydrochloride benzoic acid cocrystal was found to have an acceptable melting point, about 104° C., as shown by the DSC trace in FIG. 21. Its dissolution rate was found to be intermediate between those of the 1:1 zotepine hydrochloride benzoic acid cocrystal and zotepine hydrochloride (FIG. 25), which provides the ability to further tune the dissolution rate of the drug to the desired level based on the therapeutic use and specific formulation desired. The equilibrium solubility could not be determined with certainty, as it was shown by XRPD analysis of remaining solids following dissolution experiments that the 2:1 zotepine hydrochloride benzoic acid cocrystal converts to the 2:1 zotepine hydrochloride benzoic acid cocrystal in the presence of water.

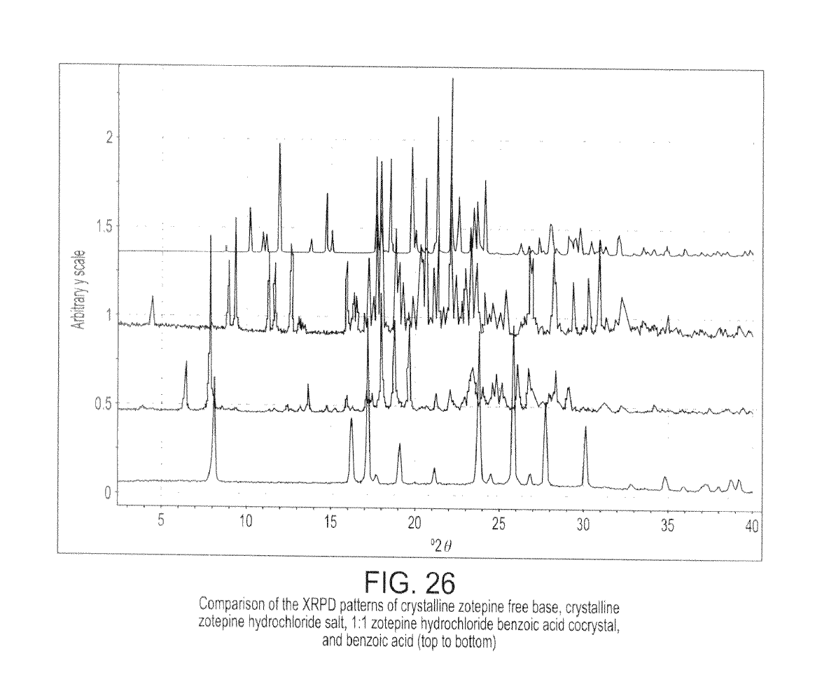

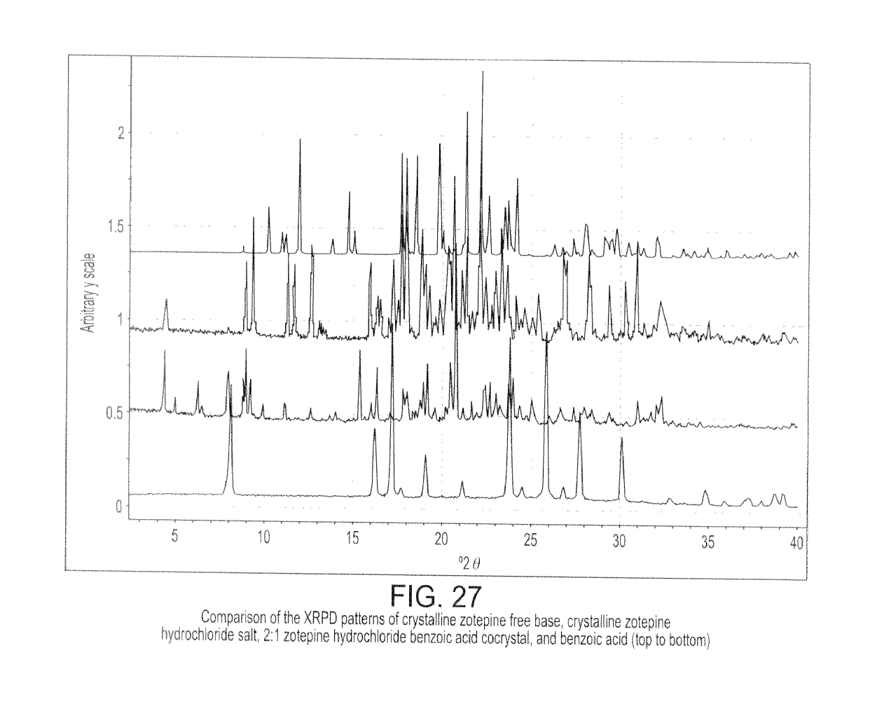

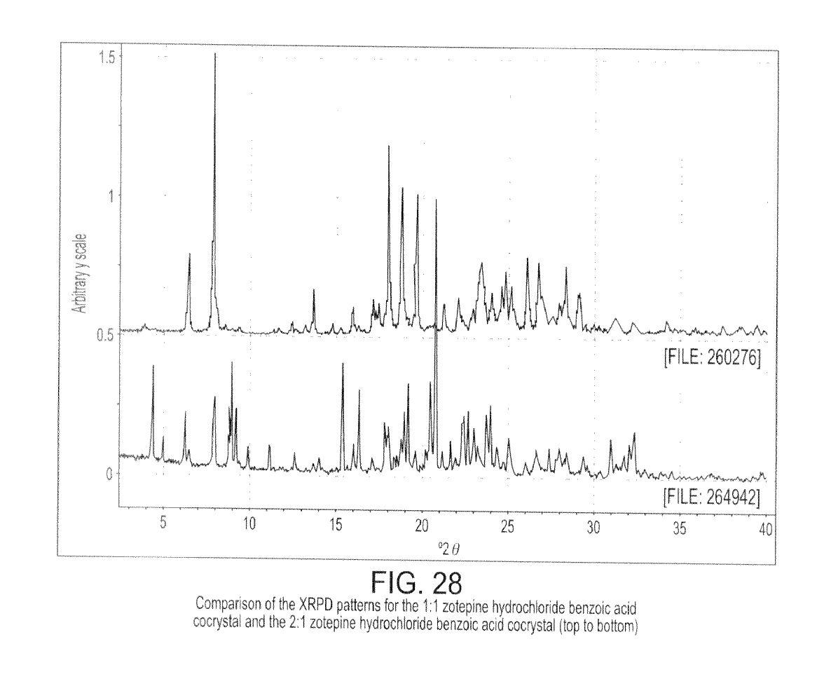

The formation of the 1:1 zotepine hydrochloride benzoic acid cocrystal and of the 2:1 zotepine hydrochloride benzoic acid cocrystal can be seen from comparisons of the physical characteristics of their components. For example, FIG. 26 compares the XRPD patterns of crystalline zotepine free base, crystalline zotepine hydrochloride salt, 1:1 zotepine hydrochloride benzoic acid cocrystal, and benzoic acid. FIG. 27 similarly compares the XRPD patterns of crystalline zotepine free base, crystalline zotepine hydrochloride salt, 2:1 zotepine hydrochloride benzoic acid cocrystal, and benzoic acid. A comparison of the XRPD patterns for the 1:1 zotepine hydrochloride benzoic acid cocrystal and the 2:1 zotepine hydrochloride benzoic acid cocrystal is shown in FIG. 28, where the top pattern is the 1:1 zotepine hydrochloride benzoic acid cocrystal and the bottom pattern is the 2:1 zotepine hydrochloride benzoic acid cocrystal.

### Pharmaceutical Compositions and Methods of Treatment

The crystalline forms of zotepine hydrochloride of the invention possess the same pharmacological activity as zotepine and are useful for treating central nervous system conditions such as those discussed above, especially schizophrenia, psychosis, and bipolar disorder. Central nervous system conditions which are psychoses or may be associated with psychotic features include, but are not limited to the psychotic disorders which have been characterized in the DSM-IV-TR. Diagnostic and Statistical Manual of Mental Disorders. Revised, 4^{th} Ed., Text Revision (2000). See also DSM-IV, Diagnostic and Statistical Manual of Mental Disorders 4^{th} Ed., (1994). The DSM-IV and DSM-IV-TR were prepared by the Task Force on Nomenclature and Statistics of the American Psychiatric Association, and provide descriptions of diagnostic categories. The skilled artisan will recognize that there are alternative nomenclatures, nosologies, and classification systems for central nervous system conditions such as those discussed above and that these systems evolve with medical scientific progress. Further examples of pathologic conditions associated with psychosis that may be treated with the compounds of the invention include, but are not limited to, schizophrenia, schizophreniform disorder, schizoaffective disorder, delusional disorder, brief psychotic disorder, shared psychotic disorder, psychotic disorder due to a general medical condition, substance-induced psychotic disorder, schizotypical, schizoid, paranoid personality disorder, and psychotic disorder-not other specified, see DSM-IV, Section: Schizophrenia and Other Psychotic Disorders, pages 273 to 316. The crystalline forms of zotepine hydrochloride described here are also useful in treating the negative symptoms and the cognitive symptoms associated with such disorders, including but not limited to, psychological conditions such as schizophrenia and other psychotic disorders.

The crystalline forms of zotepine hydrochloride according to the invention are also useful in treating depression and mood disorders found in the DSM-IV, Diagnostic and Statistical Manual of Mental Disorders 4^{th} Ed., (1994) Section: Mood Disorders, pages 317 to 392. Disorders include, but are not limited to, mood disorders such as major depressive episodes, manic episode, mixed episode, hypomanic episode; depressive disorders such as major depressive disorder, dysthymic disorder, depressive disorder not otherwise specified; bipolar disorders such as bipolar I disorder, bipolar II disorder, cyclothymic disorder, bipolar disorder not otherwise specified; other mood disorders such as mood disorder due to general medical conditions, substance-induced mood disorder, mood disorder not otherwise specified; and mood disorders with mild, moderate, severe without psychotic features, severe with psychotic features, in partial remission, in full remission, with catatonic features, with melancholic features, with atypical features, with postpartum onset.

The crystalline forms of zotepine hydrochloride according to the invention may also be used to treat depressive episodes associated with bipolar disorders, treatment of manic episodes associated with bipolar disorders such as, but not limited to, the treatment of the acute manic episodes associated with bipolar I disorder, and in the maintenance treatment of bipolar disorder to prevent recurrence of depressive or manic episodes. They are useful in treating cognitive disorders, age-related cognitive disorder, mild cognitive impairment, postconcussional disorder, mild neurocognitive disorder, anxiety (particularly including generalized anxiety disorder, panic disorder, obsessive compulsive disorder, social anxiety disorder, social phobia, and post-traumatic stress disorder), and migraine (including migraine headache).

The crystalline forms of zotepine hydrochloride according to the invention are also useful in treating substance withdrawal (including substances such as opiates, nicotine, tobacco products, alcohol, benzodiazepines, cocaine, sedatives, hypnotics, caffeine, etc.). Other conditions that may be treated with the compounds of the present invention include, but are not limited to, dementia, dementia with behavioral disturbances, movement disorders, personality disorders, borderline personality disorder, Huntington's Disease, behavioral and psychological symptoms of dementia, pain, gout, conduct disorder, autism and autism spectrum disorders, attention deficit hyperactivity disorder, insomnia, sleep disorders, pervasive development disorders, eating disorders, premenstrual dysphoric disorder, tic disorders, sexual dysfunction, delirium, emesis, substance related disorders, impulse-control disorders, postpsychotic depressive disorder of schizophrenia, simple deteriorative disorder (simple schizophrenia), minor depressive disorder, recurrent brief depressive disorder, and mixed anxiety-depressive disorder.

As discussed, the invention relates to pharmaceutical compositions comprising a therapeutically effective amount of crystalline zotepine hydrochloride, of a 1:1 zotepine hydrochloride benzoic acid cocrystal, or of a 2:1 zotepine hydrochloride benzoic acid cocrystal of the invention and a pharmaceutically acceptable carrier (also known as a pharmaceutically acceptable excipient). The crystalline zotepine hydrochloride and zotepine hydrochloride benzoic acid cocrystals of the invention have the same pharmaceutical activity as previously reported for zotepine. Pharmaceutical compositions for the treatment of those conditions or disorders contain a therapeutically effective amount of crystalline zotepine hydrochloride, a 1:1 zotepine hydrochloride benzoic acid cocrystal, or a 2:1 zotepine hydrochloride benzoic acid cocrystal of the invention, as appropriate, for treatment of a patient with the particular condition or disorder. A “therapeutically effective amount” of a crystalline form of zotepine hydrochloride according to the invention (discussed here concerning the pharmaceutical compositions) refers to an amount of a therapeutic agent to treat or prevent a condition treatable by administration of a composition of the invention. That amount is the amount sufficient to exhibit a detectable therapeutic or preventative or ameliorative effect. The effect may include, for example, treatment or prevention of the conditions listed herein. The actual amount required for treatment of any particular patient will depend upon a variety of factors including the disorder being treated and its severity; the specific pharmaceutical composition employed; the age, body weight, general health, sex and diet of the patient; the mode of administration; the time of administration; the route of administration; and the rate of excretion of zotepine; the duration of the treatment; any drugs used in combination or coincidental with the specific compound employed; and other such factors well known in the medical arts. These factors are discussed in Goodman and Gilman's “The Pharmacological Basis of Therapeutics”, Tenth Edition, A. Gilman, J. Hardman and L. Limbird, eds., McGraw-Hill Press, 155-173, 2001, which is incorporated herein by reference.

A pharmaceutical composition of the invention may be any pharmaceutical form which contains crystalline zotepine hydrochloride, a 1:1 zotepine hydrochloride benzoic acid cocrystal, or a 2:1 zotepine hydrochloride benzoic acid cocrystal according to the invention. Depending on the type of pharmaceutical composition, the pharmaceutically acceptable carrier may be chosen from any one or a combination of carriers known in the art. The choice of the pharmaceutically acceptable carrier depends upon the pharmaceutical form and the desired method of administration to be used. For a pharmaceutical composition of the invention, that is one having crystalline zotepine hydrochloride, a 1:1 zotepine hydrochloride benzoic acid cocrystal, or a 2:1 zotepine hydrochloride benzoic acid cocrystal of the invention, a carrier should be chosen that maintains its crystalline form. In other words, the carrier should not substantially alter the crystalline form of the crystalline zotepine hydrochloride, 1:1 zotepine hydrochloride benzoic acid cocrystal, or 2:1 zotepine hydrochloride benzoic acid cocrystal of the invention. Nor should the carrier be otherwise incompatible with zotepine itself, crystalline zotepine hydrochloride, the 1:1 zotepine hydrochloride benzoic acid cocrystal, or the 2:1 zotepine hydrochloride benzoic acid cocrystal of the invention, such as by producing any undesirable biological effect or otherwise interacting in a deleterious manner with any other component(s) of the pharmaceutical composition.

The pharmaceutical compositions of the invention are preferably formulated in unit dosage form for ease of administration and uniformity of dosage. A “unit dosage form” refers to a physically discrete unit of therapeutic agent appropriate for the patient to be treated. It will be understood, however, that the total daily dosage of the crystalline zotepine hydrochloride, 1:1 zotepine hydrochloride benzoic acid cocrystal, or 2:1 zotepine hydrochloride benzoic acid cocrystal of the invention and its pharmaceutical compositions according to the invention will be decided by the attending physician within the scope of sound medical judgment.

Because the crystalline form of crystalline zotepine hydrochloride, 1:1 zotepine hydrochloride benzoic acid cocrystal, or 2:1 zotepine hydrochloride benzoic acid cocrystal of the invention is more easily maintained during their preparation, solid dosage forms are a preferred form for the pharmaceutical composition of the invention. Solid dosage forms for oral administration include capsules, tablets, pills, powders, and granules. Tablets are particularly preferred. The active ingredient may be contained in a solid dosage form formulation that provides quick release, sustained release or delayed release after administration to the patient. In such solid dosage forms, the active compound is mixed with at least one inert, pharmaceutically acceptable carrier such as sodium citrate or dibasic calcium phosphate. The solid dosage form may also include one or more of: a) fillers or extenders such as starches, lactose, sucrose, glucose, mannitol, and silicic acid; b) binders such as, for example, carboxymethylcellulose, alginates, gelatin, polyvinylpyrrolidinone, sucrose, and acacia; c) humectants such as glycerol; d) disintegrating agents such as agar, calcium carbonate, potato or tapioca starch, alginic acid, certain silicates, and sodium carbonate; e) dissolution retarding agents such as paraffin; absorption accelerators such as quaternary ammonium compounds; g) wetting agents such as, for example, cetyl alcohol and glycerol monostearate; h) absorbents such as kaolin and bentonite clay; and i) lubricants such as talc, calcium stearate, magnesium stearate, solid polyethylene glycols, and sodium lauryl sulfate. The solid dosage forms may also comprise buffering agents. They may optionally contain opacifying agents and can also be of a composition that they release the active ingredient(s) only, or preferentially, in a certain part of the intestinal tract, optionally, in a delayed manner. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Solid dosage forms of pharmaceutical compositions of the invention can also be prepared with coatings and shells such as enteric coatings and other coatings well known in the pharmaceutical formulating art.

The crystalline zotepine hydrochloride, 1:1 zotepine hydrochloride benzoic acid cocrystal, or 2:1 zotepine hydrochloride benzoic acid cocrystal of the invention can be in a solid micro-encapsulated form with one or more carriers as discussed above. Microencapsulated forms may also be used in soft and hard-filled gelatin capsules with carriers such as lactose or milk sugar as well as high molecular weight polyethylene glycols and the like.

The crystalline zotepine hydrochloride, 1:1 zotepine hydrochloride benzoic acid cocrystal, or 2:1 zotepine hydrochloride benzoic acid cocrystal may also be used in the preparation of non-solid formulations, e.g., injectables and patches, of zotepine. Such non-solid formulations are known in the art. In anon-solid formulation, the crystalline form is, generally speaking, not maintained. For example, the crystalline form may be dissolved in a liquid carrier. In this case, the crystalline forms of the invention represent intermediate forms of zotepine used in the preparation of the non-solid formulation. The crystalline forms of the invention provide advantages of handling stability and purity to the process of making such formulations.

The invention also relates to the treatment of central nervous system disorders such as those discussed above. The invention provides a method for treating of central nervous system disorders using, by administering to mammals, crystalline zotepine hydrochloride, a 1:1 zotepine hydrochloride benzoic acid cocrystal, or a 2:1 zotepine hydrochloride benzoic acid cocrystal according to the invention, or a pharmaceutical composition containing one of them, in an amount sufficient to treat or prevent a condition treatable by administration of a composition of the invention. That amount is the amount sufficient to exhibit a detectable therapeutic or preventative or ameliorative effect. The effect may include, for example, treatment or prevention of the conditions listed herein. These crystalline forms and pharmaceutical compositions containing them may, according to the invention, be administered using any amount, any form of pharmaceutical composition and any route of administration effective for the treatment. After formulation with an appropriate pharmaceutically acceptable carrier in a desired dosage, as known by those of skill in the art, the pharmaceutical compositions of this invention can be administered to humans and other animals orally, rectally, or topically (as by powders or other solid form-based topical formulations). In certain embodiments, the crystalline zotepine hydrochloride, a 1:1 zotepine hydrochloride benzoic acid cocrystal, or a 2:1 zotepine hydrochloride benzoic acid cocrystal according to the invention may be administered at dosage levels of about 0.001 mg/kg to about 50 mg/kg, from about 0.01 mg/kg to about 25 mg/kg, or from about 0.1 mg/kg to about 10 mg/kg of subject body weight per day, one or more times a day, to obtain the desired therapeutic effect. It will also be appreciated that dosages smaller than 0.001 mg/kg or greater than 50 mg/kg (for example 50-100 mg/kg) can be administered to a subject. As discussed above, the amount required for treatment of a particular patient will depend upon a variety of factors including the disorder being treated and its severity; the specific pharmaceutical composition employed; the age, body weight, general health, sex and diet of the patient; the mode of administration; the time of administration; the route of administration; and the rate of excretion of zotepine; the duration of the treatment; any drugs used in combination or coincidental with the specific compound employed; and other such factors well known in the medical arts. And, as also discussed, the pharmaceutical composition of the crystalline zotepine hydrochloride, a 1:1 zotepine hydrochloride benzoic acid cocrystal, or a 2:1 zotepine hydrochloride benzoic acid cocrystal may be administered as a unit dosage form.

### EXAMPLES

Example 1 describes the characterization of crystalline benzoic acid. Example 2 describes the characterization of crystalline zotepine free base. Example 3 describes the preparation and characterization of crystalline zotepine hydrochloride. Example 4 describes the preparation and characterization of the 1:1 zotepine hydrochloride benzoic acid cocrystal, and Example 5, the preparation and characterization of the 2:1 zotepine hydrochloride benzoic acid cocrystal. The following methods and instruments were used to characterize these crystalline forms.

One of skill in the art would appreciate that certain analytical techniques, such as, for example, XRPD, ¹H-NMR, DSC, TGA, and Raman, will not produce exactly the same results every time due to, for example, instrumental variation, sample preparation, scientific error, etc. By way of example only, XRPD results (i.e. peak locations, intensities, and/or presence) may vary slightly from sample to sample, despite the fact that the samples are, within accepted scientific principles, the same form, and this may be due to, for example, preferred orientation or varying solvent or water content. It is well within the ability of those skilled in the art, looking at the data as a whole, to appreciate whether such differences indicate a different form, and thus determine whether analytical data being compared to those disclosed herein are substantially similar. In this regard, and as is commonly practiced within the scientific community, it is not intended that the exemplary analytical data of the crystalline forms of zotepine hydrochloride according to the invention disclosed here be met literally in order to determine whether comparative data represent the same form as those disclosed and claimed herein, such as, for example, whether each and every peak of an exemplary XRPD pattern in comparative data, in the same location, and/or of the same intensity. Rather it is intended that those of skill in the art, using accepted scientific principles, will make a determination regarding whether comparative analytical data represent the same or a different form.

X-Ray Powder Diffraction (XRPD): Samples were analyzed using a PANalytical X'Pert Pro diffractometer. The specimen was analyzed using Cu radiation produced using an Optix long fine-focus source. An elliptically graded multilayer mirror was used to focus the Cu Kα X-rays of the source through the specimen and onto the detector. The specimen was sandwiched between 3-micron thick films, analyzed in transmission geometry, and rotated to optimize orientation statistics. A beam-stop and in some cases a helium purge were used to minimize the background generated by air scattering. Soller slits were used for the incident and diffracted beams to minimize axial divergence. Diffraction patterns were collected using a scanning position-sensitive detector (X'Celerator) located 240 mm from the specimen. Prior to the analysis a silicon specimen (NIST standard reference material 640 c) was analyzed to verify the position of the silicon 111 peak.

Single Crystal X-ray Diffraction (Data Collection): A colorless needle of 1:1 zotepine hydrochloride benzoic acid cocrystal (C₂₅H₂₅Cl₂NO₃S[Cl,C₁₈H₁₉ClNOS,C₇H₆O₂]) having approximate dimensions of 0.38×0.20×0.05 mm, was mounted on a glass fiber in random orientation. Preliminary examination and data collection were performed with Mo K_{α} radiation (λ=0.71073 Å) on a Nonius KappaCCD diffractometer equipped with a graphite crystal, incident beam monochromator. Refinements were performed on an LINUX PC using SHELX97 (Sheldrick, G. M. Acta Cryst., 2008, A64, 112). Cell constants and an orientation matrix for data collection were obtained from least-squares refinement using the setting angles of 4734 reflections in the range 2°<θ<25°. The refined mosaicity from Denzo/Scalepack was not determined so no assessment of the crystal quality can be made. The space group was determined by the program ASPEN (McArdle, P. C J. Appl. Cryst. 1996, 29, 306). From the systematic presence of the following conditions: hk0: h+k=2n; 0kl: l=2n; h0l: l=2n, and from subsequent least-squares refinement, the space group was determined to be Pccn (no. 56). The data were collected to a maximum 2θ value of 50.07°, at a temperature of 150±1 K.

Single Crystal X-ray Diffraction (Data Reduction): Frames were integrated with DENZO-SMN. A total of 4734 reflections were collected, of which 4154 were unique. Lorentz and polarization corrections were applied to the data. The linear absorption coefficient is 0.380 m⁻¹ for Mo K_{α}radiation. An empirical absorption correction using SCALEPACK was applied. Intensities of equivalent reflections were averaged. The agreement factor for the averaging was not reported for this data set.

Single Crystal X-ray Diffraction (Structure Solution and Refinement): The structure was solved by direct methods using SIR2004 (Burla, M. C., Caliandro, R., Camalli, M,. Carrozzini, B., Cascarano, G. L., De Caro, L., Giacovazzo, C., Polidori, G., and Spagna, R., J. Appl. Cryst. 2005, 38, 381). The remaining atoms were located in succeeding difference Fourier syntheses. Hydrogen atoms were included in the refinement but restrained to ride on the atom to which they are bonded. The structure was refined in full-matrix least-squares by minimizing the function:

Σw(|Fₒ|²−|F_{c}|²)²

The weight w is defined as 1/[σ²(Fₒ²)+(0.0956P)²+(3.3431P)], where P=(Fₒ²+2F_{c}²)/3. Scattering factors were taken from the “International Tables for Crystallography” (International Tables for Crystallography, Vol. C, Kluwer Academic Publishers: Dordrecht, The Netherlands, 1992, Tables 4.2.6.8 and 6.1.1.4). Of the 4154 reflections used in the refinements, only the reflections with Fₒ²>2σ(Fₒ²) were used in calculating R. A total of 2698 reflections were used in the calculation. The final cycle of refinement included 299 variable parameters and converged (largest parameter shift was <0.01 times its estimated standard deviation) with unweighted and weighted agreement factors of:

R=Σ|Fₒ−F_{c}|/ΣFₒ=0.063

R_{w}={square root over ((Σw(Fₒ²−F_{c}²)²/Σw((fₒ²)²))}=0.165

The standard deviation of an observation of unit weight was 1.061. The highest peak in the final difference Fourier had a height of 0.55 e/Å³. The minimum negative peak had a height of −0.43 e/Å³.

Single Crystal X-ray Diffraction (ORTEP and Packing Diagrams): The ORTEP diagram was prepared using ORTEP III (Johnson, C. K. ORTEPIII, Report ORNL-6895, Oak Ridge National Laboratory, TN, U.S.A. 1996. OPTEP-3 for Windows V1.05, Farrugia, L. J., J. Appl. Cryst. 1997, 30, 565) program within the PLATON (Spek, A. L. PLUTON. Molecular Graphics Program. Univ. of Ultrecht, The Netherlands 1991. Spek, A. L. Acta Crystallogr., 1990, A46, C34) software package. Atoms are represented by 50% probability anisotropic thermal ellipsoids. Packing diagrams were prepared using CAMERON (Watkin, D. J.; Prout, C. K.; Pearce, L. J. CAMERON, Chemical Crystallography Laboratory, University of Oxford, Oxford, 1996) modeling software. Assessment of chiral centers, void calculations and additional figures were performed with the PLATON software package. Additional figures were also generated with the Mercury 1.5 (Macrae, C. F. Edgington, P. R. McCabe, P. Pidcock, E. Shields, G. P. Taylor, R. Towler M. and van de Streek, J.; J. Appl. Cryst., 2006, 39, 453-457) visualization package. Hydrogen bonding is represented as dashed lines.

Differential Scanning calorimetry: Differential scanning calorimetry (DSC) was performed using a TA Instruments differential scanning calorimeter 2920. The sample was placed in an aluminum DSC pan, and the weight accurately recorded. The pan was covered with a lid, then crimped and analyzed up to a final temperature of 250° C. Indium metal was used as the calibration standard. Reported temperatures are at the transition maxima.

Thermogravimetric analysis: Thermogravimetric (TG) analyses were performed using a TA Instruments 2950 thermogravimetric analyzer. Each sample was placed in an aluminum sample pan and inserted into the TG furnace. The furnace was first equilibrated at 25° C., then heated under nitrogen at a rate of 10° C./min, up to a final temperature of either 300 or 350° C. Nickel and Alumel™ were used as the calibration standards.

Dispersive Raman: Dispersive Raman spectra were acquired on a Renishaw Mk1 Ramascope model 1000 equipped with a Leica DM LM microscope. A 50× objective was used for the analysis. The excitation wavelength was 785 nm and the laser was at 50% power. A continuous grating scan from 3200 to 100 cm⁻¹ was used with an exposure time of 10 seconds and high gain. The samples were analyzed at a spectral resolution of 4 cm⁻¹. The samples were prepared for analysis by placing particles onto a gold mirror. The instrument was calibrated with a silicon wafer standard and a neon emission lamp.

FT-Raman: FT-Raman spectra were acquired on an FT-Raman 960 spectrometer (Thermo Nicolet). This spectrometer uses an excitation wavelength of 1064 nm. Approximately 1.0-1.5 W of Nd:YVO₄ laser power was used to irradiate the sample. The Raman spectra were measured with a gennanium (Ge) detector. The samples were prepared for analysis by placing the material in a glass tube and positioning the tube in a gold-coated tube holder in the accessory. A total of 256 sample scans were collected from 100-3600 cm⁻¹ at a spectral resolution of 4 cm⁻¹. using Happ-Genzel apodization. Wavelength calibration was performed using sulfur and cyclohexane.

¹H Numclear Magnetic Resonance (NMR): The solution phase ¹H NMR spectra were obtained on a Varian INOVA-400 spectrometer (¹H Larmor frequency 399.800 MHz) at ambient temperature. Samples were prepared for ¹H NMR spectroscopy as ˜5-50 mg solutions in deuterated DMSO with tetramethylsilane (DMSO-d6/TMS). Spectra were referenced to internal tetramethylsilane at 0.0 ppm.

Equilibrium Solubility—UV Measurement: Equilibrium solubility was determined in water using ambient-temperature slurry experiments. Samples were prepared with excess solids and agitated on a wheel for at least 3 days. Remaining solids were separated from the mixture by centrifugation. The clear supernatant was pipeted to a separate container and the concentration determined through ultraviolet (UV) spectrophotometry, diluting the sample with additional water if necessary. An analytical wavelength of 302 nm was chosen to avoid potential interference from benzoic acid. Equivalent zotepine hydrochloride salt concentrations were calculated from the Beer's Law plot generated front zotepine hydrochloride salt aqueous standards and adjusted to account for the stoichiometry of the solids utilized. Retained solids were analyzed by X-ray powder diffraction, if sufficient solids were present. Concentrations of aqueous solutions of BNV-218 were determined through ultraviolet absorbance.

Ultraviolet spectrophotometry: Solutions were analyzed using a Cary 50 dual-beam spectrophotometer. They were analyzed at ambient temperature in a 1.000-cm quartz cuvette. Scans at 600 nm/min in the range of 800-200 nm were performed to determine an optimal wavelength for concentration measurement. The cuvette was washed with methanol, followed by water, and the detector was then zeroed prior to analysis of each sample. Wavelength calibration was performed using holmium oxide. The photometric accuracy was verified by measuring the intensity of the light at the detector when filters of known optical density were placed in the path of the beam.

Intrinsic Dissolution: Pellets of approximately 200 mg were pressed at 3000 lbs. for 1 minute in a standard Woods apparatus, with a surface area of 0.5 cm². Three pellets were tested for each material, testing one material at a time. The samples were rotated in a VanKel dissolution apparatus, with automated sampling, at 50 RPM in 900 mL of water at 25° C. Aliquots were taken every two minutes and not filtered prior to analysis. Concentrations were determined through UV absorbance at approximately 302 nm, to avoid potential interference from benzoic acid. Equivalent hydrochloride salt concentrations were calculated from the Beer's Law plot generated from zotepine hydrochloride salt aqueous standards.

To determine the rate of dissolution of each material, a plot was generated of the absorbance over time for each vessel. From this plot, a linear region was chosen from the initial dissolution period of each material: starting at the first time point when the measured absorbance exceeded the minimum measured absorbance value in the Beer's Law relationship generated from zotepine hydrochloride salt aqueous standards. The end of the linear region was chosen as either 30 minutes after the first time point, or at the maximum recorded absorbance value that falls within the Beer's Law Relationship. Equivalent hydrochloride salt concentrations were plotted versus time for the regions. A straight-line was fit to the data for each vessel. The slope of these lines provides the dissolution rate for each of the materials, expressed as [μg/mL]/min. The mean of the three rates is the reported rate for each material, expressed as equivalent hydrochloride salt concentration, for direct comparison of the materials. The rates were not normalized for the surface area of the pellet.

### Example 1

### Characterization of Crystalline Benzoic Acid

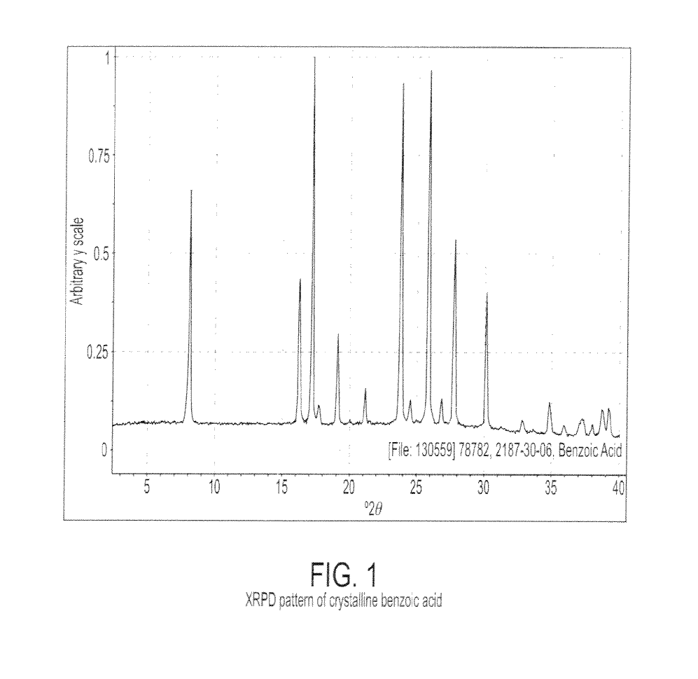

Crystalline benzoic acid was obtained from Aldrich. Crystalline benzoic acid was characterized by XRPD using a PANalytical X'Pert Pro diffractometer. The measurement conditions are reported in Table 1. FIG. 1 is a representative XRPD pattern of crystalline benzoic acid. Table 2 reports the peaks identified in the XRPD pattern.

TABLE 1
XRPD Measurement Conditions for Crystalline Benzoic Acid

| Condition | Value |
|---|---|
| Instrument | Panalytical X-Pert Pro |
| | MPD PW3040 Pro |
| X-ray tube | Cu (1.54059 Å) |
| Voltage | 45 kV |
| Amperage | 40 mA |
| Scan range | 1.01-39.98 °2θ |
| Step size | 0.017 °2θ |
| Collection time | 1940 s |
| Scan speed | 1.2°/min |
| Slit | DS: ½°; SS: ¼° |
| Revolution time | 0.5 s |
| Mode | Transmission |

TABLE 2
Peak Positions of the XRPD Pattern for Crystalline Benzoic Acid

| Degrees 2θ | Intensity % (I/Io) |
|---|---|
| 8.1 ± 0.2 | 63 |
| 16.2 ± 0.2 | 39 |
| 17.2 ± 0.2 | 100 |
| 17.7 ± 0.2 | 6 |
| 19.1 ± 0.2 | 25 |
| 21.2 ± 0.2 | 10 |
| 23.8 ± 0.2 | 91 |
| 24.5 ± 0.2 | 7 |
| 25.9 ± 0.2 | 93 |
| 26.9 ± 0.2 | 7 |
| 27.8 ± 0.2 | 51 |
| 30.2 ± 0.2 | 36 |

### Example 2

### Characterization of Crystalline Zotepine Free Base

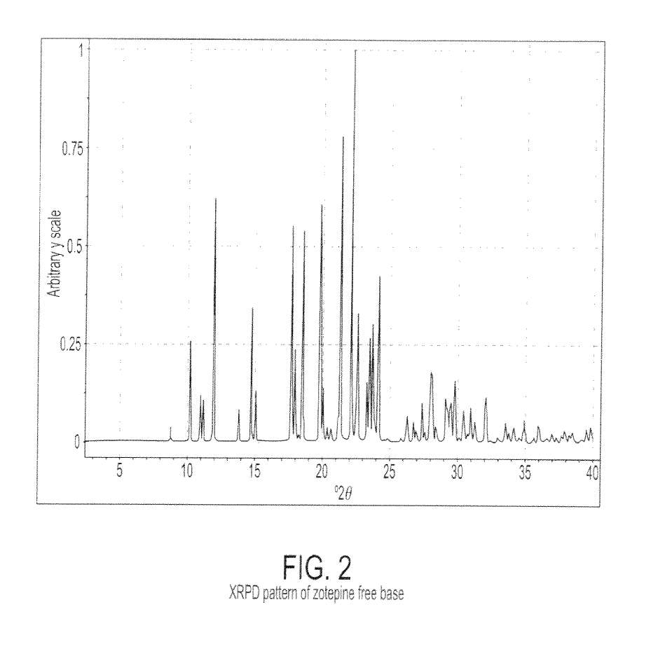

Crystalline Zotepine free base was obtained from Hallochem Pharma, Chongqing, China. Crystalline zotepine free base was characterized by XRPD using a PANalytical X'Pert Pro diffractometer. The measurement conditions are reported in Table 3. The XRPD pattern is shown in FIG. 2. Table 4 reports the peaks identified in the XRPD pattern.

TABLE 3
XRPD Measurement Conditions for
Crystalline Zotepine Free Base.

| Condition | Value |
|---|---|
| Instrument | Panalytical X-Pert Pro |
| | MPD PW3040 Pro |
| X-ray tube | Cu (1.54059 Å) |
| Voltage | 45 kV |
| Amperage | 40 mA |
| Scan range | 1.01-39.98 °2θ |
| Step size | 0.017 °2θ |
| Collection time | 1936 s |
| Scan speed | 1.2°/min |
| Slit | DS: ½°; SS: ¼° |
| Revolution time | 0.5 s |
| Mode | Transmission |

TABLE 4
Peak Positions of the XRPD Pattern
for Crystalline Zotepine Free Base

| Degrees 2θ | Intensity % (I/Io) |
|---|---|
| 8.8 ± 0.2 | 4 |
| 10.3 ± 0.2 | 26 |
| 11.0 ± 0.2 | 12 |
| 11.2 ± 0.2 | 11 |
| 12.0 ± 0.2 | 63 |
| 13.8 ± 0.2 | 8 |
| 14.8 ± 0.2 | 35 |
| 15.1 ± 0.2 | 13 |
| 17.7 ± 0.2 | 56 |
| 18.0 ± 0.2 | 24 |
| 18.3 ± 0.2 | 2 |
| 18.6 ± 0.2 | 54 |
| 19.8 ± 0.2 | 61 |
| 20.0 ± 0.2 | 14 |
| 20.4 ± 0.2 | 4 |
| 20.7 ± 0.2 | 4 |
| 21.2 ± 0.2 | 7 |
| 21.3 ± 0.2 | 78 |
| 22.2 ± 0.2 | 100 |
| 22.6 ± 0.2 | 33 |
| 23.3 ± 0.2 | 15 |
| 23.5 ± 0.2 | 27 |
| 23.7 ± 0.2 | 31 |
| 23.9 ± 0.2 | 6 |
| 24.1 ± 0.2 | 43 |

FIG. 3 depicts representative DSC/TGA analyses of crystalline zotepine free base. The DSC showed a major endotherm with peak maximum at 92° C., corresponding to the previously reported melting point of 90-91° C. (Merck Index, 13^{th} edition). The TGA showed a 0.16% weight loss up to 150° C.

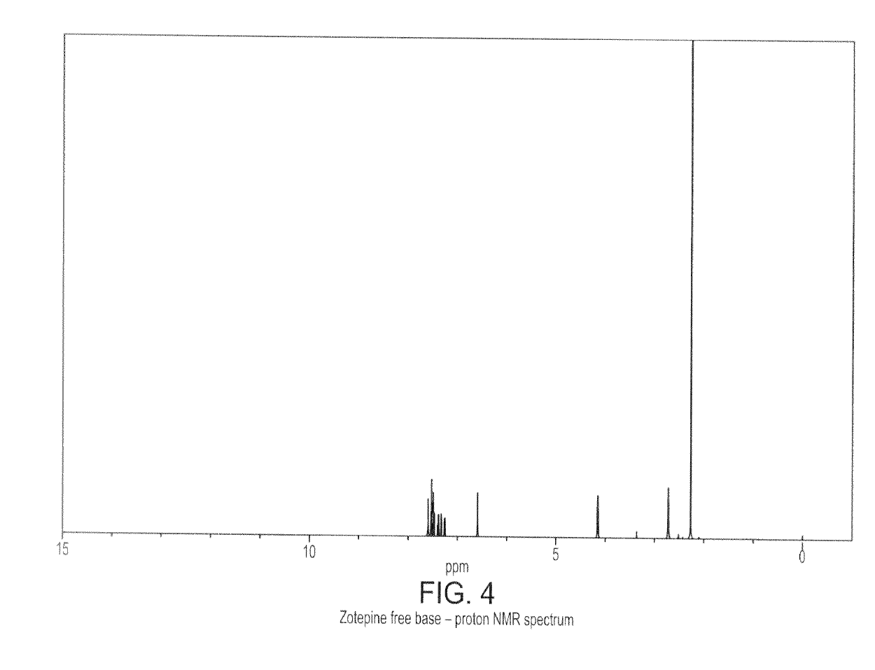

FIG. 4 depicts the proton NMR spectrum of zotepine free base in deuterated DMSO. Table 5 lists the observed peaks and their integration.

TABLE 5
¹H NMR of Zotepine Free Base

| | peak | | coupling | |
|---|---|---|---|---|
| | position | | constant | number of |
| protons | (ppm) | multiplicity | (Hz) | protons |
| CH₃ | 2.28 | singlet | — | 6 |
| CH₂N | 2.72 | triplet | 5.8 | 2 |
| CH₂O | 4.15 | triplet | 5.8 | 2 |
| C═CH | 6.60 | singlet | — | 1 |
| aromatic | 7.24-7.41 | multiplet | — | 3 |
| aromatic | 7.47-7.55 | multiplet | — | 3 |
| aromatic | 7.61 | doublet | 2.1 | 1 |

Approximate and equilibrium solubility measurements were attempted for zotepine free base, yielding negligible solubility values. Approximate solubility measurements yielded a value of less than 4 mg/mL (4 mg of zotepine free base were added to 1 mL of water, but the solids did not completely dissolve, yielding a very hazy liquid with remaining solids). Equilibrium solubility experiments to measure UV absorbance of the zotepine free base solution at 302 nm yielded negligible absorbance values near zero, which fell below the minimum absorbance value measured for the Beer's Law Plot relationship for concentration vs. absorbance generated from zotepine hydrochloride salt aqueous standards. At the end of the equilibrium solubility experiment, the largest recorded UV absorbance value for zotepine free base was 0.02, a value smaller than the inherent variability of the measurement. An equilibrium concentration value for zotepine free base was therefore not calculated, but it can be appreciated that the equilibrium concentration is negligible. The solid recovered from the solubility experiment was found by XRPD analysis to be zotepine free base.

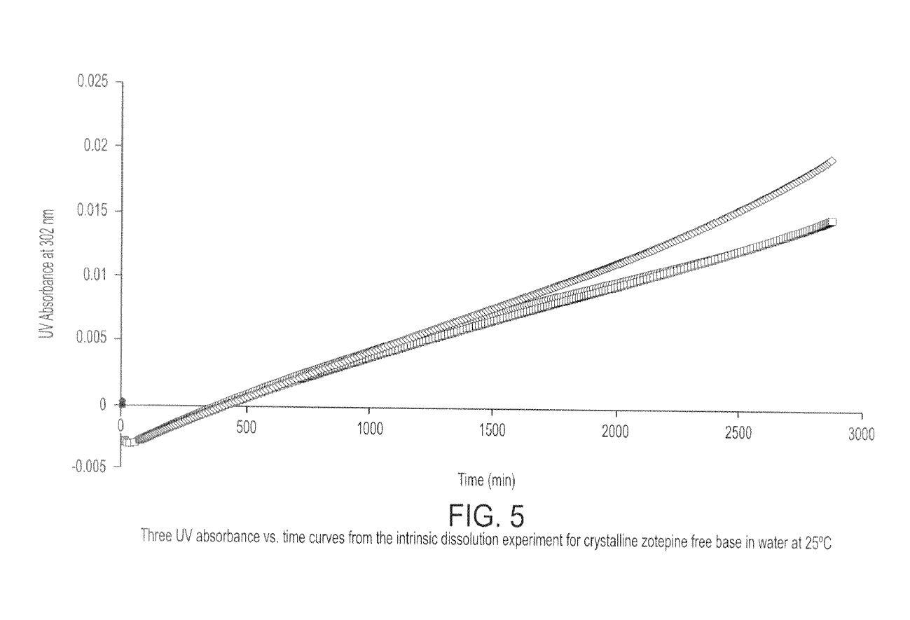

FIG. 5 depicts three UV absorbance (at 302 nm) vs. time curves for the intrinsic dissolution experiment on crystalline zotepine free base in water at 25° C. A low absorbance was observed throughout the time of the zotepine free base dissolution experiment, with a maximum absorbance of 0.02 observed when the experiment was ended at 2,880 minutes. These absorbance values fell below the minimum absorbance value measured for the Beer's Law Plot relationship generated from zotepine hydrochloride salt aqueous standards. Concentrations for the free base were therefore too low to be calculated, and the dissolution rate was therefore too low to be calculated.

### Example 3

### Preparation and Characterization of Crystalline Hydrogen Chloride Salt of Zotepine, “Crystalline Zotepine Hydrochloride”

### Example 3.1

### Preparation of Zotepine Hydrochloride

Zotepine (2008 mg, 6.051 mmol) was charged to a 250-mL round bottom flask containing a Teflon stirring bar. Diethyl ether (78 mL) was added. The mixture was stirred at 450 RPM on a Dataplate for approximately 2 minutes, producing a clear, very pale yellow solution. The pot was purged three times with nitrogen gas, then placed under a nitrogen pad. Addition of concentrated hydrochloric acid (512 μL; 6.2 mmol) was begun at 22.2° C. and completed in about one minute, with a final temperature of 23.6° C. Immediate precipitation of white solids was observed as soon as the acid addition was begun, producing a milky mixture. Stirring was continued under a nitrogen pad for about 18 hours. The mixture was filtered on a 0.2-μm Pall TF-200 PTFE membrane inside a Millipore Swinnex filter body. The solid was deliquored, then transferred to a tared 20-mL scintillation vial and dried in a vacuum oven at ambient temperature and a vacuum of approximately 30 inches mercury for approximately 3 hours. During this time, the hard, clumpy solid was manually mashed with a spatula and weighed on a balance to monitor weight loss. The sample was dried until a weight loss of 0.1% between weighings was achieved. A total of 2.028 g of solid (91% yield) was recovered.

### Example 3.2

### Characterization of Zotepine Hydrochloride

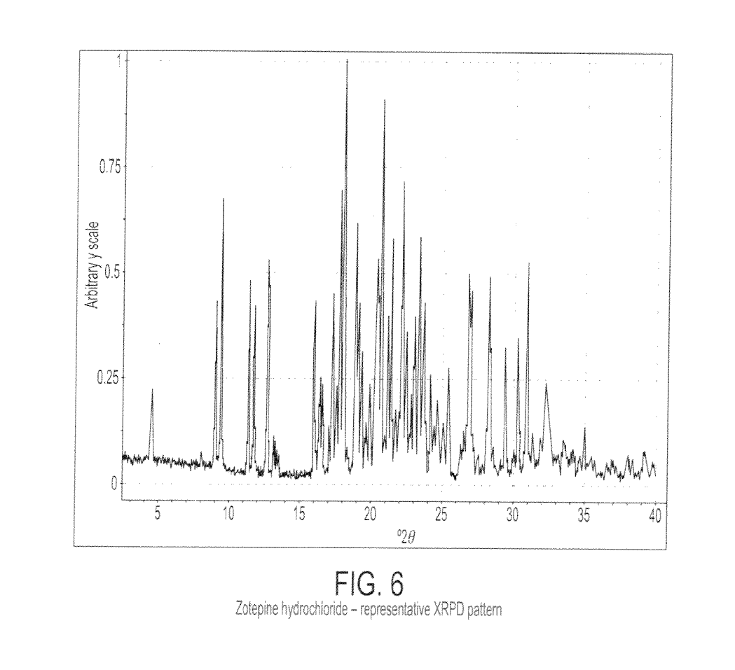

Crystalline zotepine hydrochloride was characterized by XRPD using a PANalytical X'Pert Pro diffractometer. The measurement conditions are reported in Table 6. FIG. 6 depicts a representative XRPD pattern of crystalline zotepine hydrochloride. Table 7 reports the peaks identified in the XRPD pattern. The XRPD pattern, the peaks identified in the pattern or subsets of those peaks may be used to identify crystalline zotepine hydrochloride. Peaks identified with an asterisk (*) may be considered characteristic for crystalline zotephine hydrochloride.

TABLE 6
XRPD Measurement Conditions for Crystalline
Zotepine Hydrochloride

| Condition | Value |
|---|---|
| Instrument | Panalytical X-Pert Pro |
| | MPD PW3040 Pro |
| X-ray tube | Cu (1.54059 Å) |
| Voltage | 45 kV |
| Amperage | 40 mA |
| Scan range | 1.01-39.98 °2θ |
| Step size | 0.017 °2θ |
| Collection time | 1941 s |
| Scan speed | 1.2°/min |
| Slit | DS: ½°; SS: ¼° |
| Revolution time | 0.5 s |
| Mode | Transmission |

TABLE 7
Peak Positions of the XRPD Pattern for
Crystalline Zotepine Hydrochloride

| Degrees 2θ | Intensity % (I/Io) |
|---|---|
| 4.5 ± 0.2 | 23 |
| 8.0 ± 0.2 | 8 |
| 9.0 ± 0.2 | 43 |
| 9.4* ± 0.2 | 67 |
| 11.4 ± 0.2 | 47 |
| 11.7* ± 0.2 | 42 |
| 12.7* ± 0.2 | 52 |
| 13.2 ± 0.2 | 11 |
| 13.3 ± 0.2 | 8 |
| 13.5 ± 0.2 | 6 |
| 16.0 ± 0.2 | 43 |
| 16.4 ± 0.2 | 23 |
| 16.5 ± 0.2 | 23 |
| 17.1 ± 0.2 | 14 |
| 17.3 ± 0.2 | 42 |
| 17.6 ± 0.2 | 23 |
| 17.8 ± 0.2 | 69 |
| 18.0 ± 0.2 | 100 |
| 18.3 ± 0.2 | 9 |
| 18.9 ± 0.2 | 61 |
| 19.1 ± 0.2 | 42 |
| 19.3 ± 0.2 | 31 |
| 19.7 ± 0.2 | 14 |
| 19.9 ± 0.2 | 23 |
| 20.2 ± 0.2 | 26 |
| 20.3 ± 0.2 | 53 |
| 20.7 ± 0.2 | 90 |
| 20.9 ± 0.2 | 11 |
| 21.1 ± 0.2 | 39 |
| 21.4 ± 0.2 | 57 |
| 21.7 ± 0.2 | 17 |
| 21.9 ± 0.2 | 17 |
| 22.1 ± 0.2 | 71 |
| 22.4 ± 0.2 | 34 |
| 22.7 ± 0.2 | 21 |
| 23.0 ± 0.2 | 39 |
| 23.4 ± 0.2 | 55 |
| 23.7 ± 0.2 | 42 |
| 24.2 ± 0.2 | 22 |
| 24.4 ± 0.2 | 13 |
| 24.6 ± 0.1 | 19 |

FIG. 7 depicts the DSC/TGA analyses of crystalline zotepine hydrochloride. The DSC showed a major endotherm with peak maximum at 208° C. The TGA showed a 1.4 wt. % loss from 27 to 180° C.

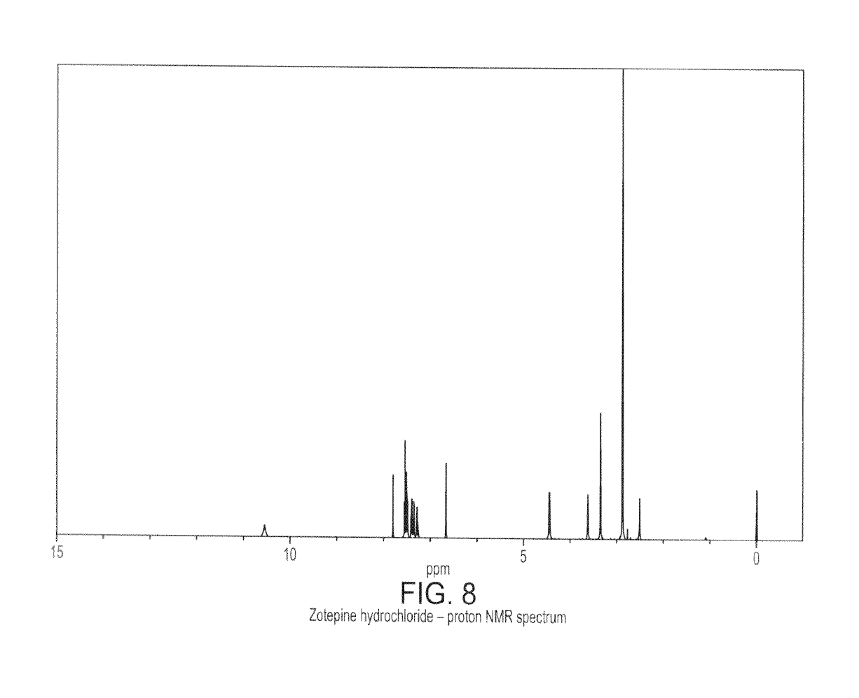

FIG. 8 depicts the proton NMR spectrum of zotepine hydrochloride. The peaks in the solution phase ¹H NMR spectrum are reported in Table 8. Formation of the hydrochloride salt is confirmed by three observations. First, large downfield shifts of the protons adjacent to the nitrogen atom were observed (N—CH₂ protons move from 2.72 ppm in the free base to 3.62 ppm in the salt; CH₃ protons move from 2.28 ppm in the free base to 2.88 ppm in the salt). Second, smaller downfield shifts in protons close to the nitrogen atom were observed (O—CH₂ protons moved from 4.15 ppm in the free base to 4.45 ppm in the salt). Third, a proton was observed to appear on the nitrogen at 10.57 ppm, which integrates to 1, in the salt. That proton is absent in the free base. Note that in deuterated DMSO, the proton on nitrogen rapidly exchanges and no coupling to neighboring protons is observed.

TABLE 8
¹H NMR Peaks for Crystalline Zotepine Hydrochloride

| | peak | | coupling | |
|---|---|---|---|---|
| | position | | constant | number of |
| protons | (ppm) | multiplicity | (Hz) | protons |
| CH₃ | 2.88 | singlet | — | 6 |
| CH₂N | 3.62 | triplet | 5.0 | 2 |
| CH₂O | 4.45 | triplet | 5.0 | 2 |
| C═CH | 6.66 | singlet | — | 1 |
| aromatic | 7.27-7.31 | multiplet | — | 3 |
| aromatic | 7.49-7.57 | multiplet | — | 3 |
| aromatic | 7.80 | doublet | 2.2 | 1 |
| NH | 10.57 | broad singlet | — | 1 |

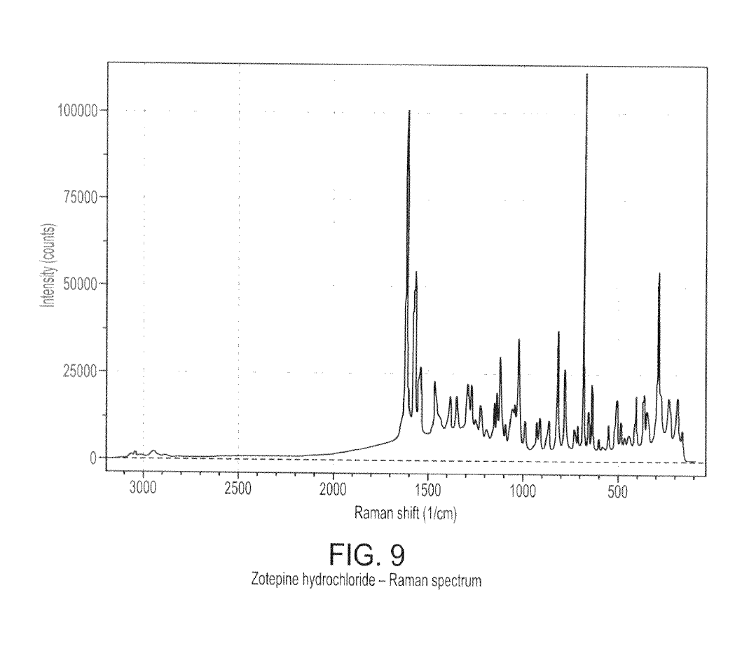

FIG. 9 depicts the Raman spectrum of crystalline zotepine hydrochloride. Table 9 reports the absorbance peaks in the Raman spectrum. The Raman spectrum, the peaks identified in the spectrum or subsets of those peaks may be used to identify crystalline zotepine hydrochloride. Peaks identified with an asterisk (*) may be considered characteristic for crystalline zotepine hydrochloride.

TABLE 9
Peaks in the Raman Spectrum of Crystalline
Zotepine Hydrochloride

| Peak position (cm⁻¹) | Rel. Intensity (%) |
|---|---|
| 286 ± 1 cm⁻¹ | 19 |
| 300 ± 1 cm⁻¹ | 49 |
| 357 ± 1 cm⁻¹ | 13 |
| 373 ± 1 cm⁻¹ | 17 |
| 413 ± 1 cm⁻¹ | 17 |
| 451 ± 1 cm⁻¹ | 7 |
| 472 ± 1 cm⁻¹ | 6 |
| 493 ± 1 cm⁻¹ | 10 |
| 515 ± 1 cm⁻¹ | 16 |
| 525 ± 1 cm⁻¹ | 9 |
| 557 ± 1 cm⁻¹ | 9 |
| 609 ± 1 cm⁻¹ | 6 |
| 645* ± 1 cm⁻¹ | 20 |
| 664 ± 1 cm⁻¹ | 13 |
| 691 ± 1 cm⁻¹ | 100 |
| 718 ± 1 cm⁻¹ | 9 |
| 736 ± 1 cm⁻¹ | 9 |
| 788* ± 1 cm⁻¹ | 24 |
| 825 ± 1 cm⁻¹ | 34 |
| 871 ± 1 cm⁻¹ | 10 |
| 881 ± 1 cm⁻¹ | 7 |
| 919 ± 1 cm⁻¹ | 11 |
| 934 ± 1 cm⁻¹ | 10 |
| 998 ± 1 cm⁻¹ | 10 |
| 1032* ± 1 cm⁻¹ | 32 |
| 1051 ± 1 cm⁻¹ | 15 |
| 1099 ± 1 cm⁻¹ | 10 |
| 1121 ± 1 cm⁻¹ | 15 |
| 1131 ± 1 cm⁻¹ | 27 |
| 1144 ± 1 cm⁻¹ | 18 |
| 1159 ± 1 cm⁻¹ | 15 |
| 1231 ± 1 cm⁻¹ | 14 |
| 1279 ± 1 cm⁻¹ | 19 |
| 1299 ± 1 cm⁻¹ | 20 |
| 1358 ± 1 cm⁻¹ | 17 |
| 1392 ± 1 cm⁻¹ | 17 |
| 1474 ± 1 cm⁻¹ | 20 |
| 1550 ± 1 cm⁻¹ | 24 |
| 1578 ± 1 cm⁻¹ | 49 |
| 1624 ± 1 cm⁻¹ | 90 |

An attempt was made to measure the aqueous solubility of zotepine hydrochloride. To a 1-dram vial was charged 99 mg of zotepine hydrochloride, and 1 mL of HPLC-grade water (obtained from Mallinckrodt) was added. The mixture was placed on a rotating wheel, with dissolution of the solids occurring in approximately 1 minute. An additional 100 mg of zotepine hydrochloride (100 mg) was therefore added, and the vial was shaken vigorously to dissolve the solids. Sonication was employed to remove suds, which had formed based on visual inspection, indicating surface activity. The aqueous concentration at this point was calculated to be ≧200 mg/mL, based on the amount of solid added.

A 0.5-mL aliquot of the resulting zotepine hydrochloride aqueous solution was then charged to a second 2-dram vial, and 50 mg of zotepine hydrochloride (50 mg) was added. A clear solution was obtained after approximately 12 minutes on the rotating wheel. An additional 50 mg zotepine hydrochloride was therefore added, with a clear, pale yellow, viscous oil being produced after approximately 35 minutes on the rotating wheel. At this point, the procedure was discontinued. The calculated aqueous concentration at this point was ≧400 mg/mL when the procedure was discontinued.

FIG. 10 depicts the intrinsic dissolution curves for crystalline zotepine hydrochloride in water. The intrinsic dissolution rate of zotepine hydrochloride was 3.9 [μg/mL]/min.

### Example 4

### Preparation and Characterization of 1:1 Zotepine Hydrochloride Benzoic Acid Cocrystal

### Example 4.1

### Preparation of 1:1 Zotepine Hydrochloride Benzoic Acid Cocrystal

Slurry Preparations: Benzoic acid (340 mg) was added to acetonitrile (4 mL). The resulting slurry was agitated for approximately 4 hours at ambient temperature and filtered through a Whatman 0.2-μm nylon disc. An aliquot of the filtrate (2.5 mL) was added to a 1-dram vial containing 30 mg of zotepine hydrochloride and the mixture was agitated for approximately 3 days at ambient temperature, producing a clear solution. Additional zotepine hydrochloride was added and the mixture was agitated for approximately 3 days at ambient temperature, again producing a clear solution. An aliquot of this solution (1 mL) was mixed with zotepine hydrochloride (50 mg) and the resulting slurry was agitated for approximately) day at ambient temperature. A slurry was still present, and was filtered through a Magna 0.22-μm nylon membrane in a Millipore Swinnex filter body, yielding isolated solids. Examination of the solid sample under a stereomicroscope revealed a mixture of chunks and plates exhibiting birefringence and extinguishment. X-ray powder diffraction analysis showed the solid to be the 1:1 zotepine hydrochloride benzoic acid cocrystal.

Symyx High Throughput Screen: Benzoic acid (612 mg) was added to methanol (50 mL) to give a 0.1 M solution. Zotepine hydrochloride (520 mg) was dissolved in methanol (10 L) and the resulting 0.14 M solution was filtered through a Pall CR-13 PTFE Acrodisc. Portions of the benzoic acid solution (90 μL each) were added to each of three microwells of a 96-well plate. Portions of the zotepine hydrochloride solution (64 μL each) were added to each microwell to give solutions containing a 1:1 molar ratio of benzoic acid to zotepine hydrochloride. Methanol was evaporated from the solutions at ambient temperature, under a vacuum of approximately 30 inches mercury, over a period of approximately 1 hour using a LabConco CentriVap Concentrator. The residues in the bottoms of the microwells had the appearance of glass. Acetone (30 μL) was added to the residue in one microwell, acetonitrile (30 μL) was added to the residue in one microwell, and 1-propanol (30 μL) was added to the residue in the third microwell. The microplate was sonicated using a MatriCal SonicMan, then left in a fume hood for a period of approximately 5 days, during which time the solvents evaporated. Solids remained in the acetone well, the acetonitrile well, and in the 1-propanol well. XRPD analyses showed that each of the three samples was the 1:1 zotepine hydrochloride benzoic acid cocrystal.

Dry Milling: Benzoic acid (25 mg, 0.21 mmol) and zotepine hydrochloride (75 mg, 0.21 mmol) were charged to an agate milling chamber along with a 5-mm diameter agate ball. The chamber was closed and agitated on a Retsch MM200 mill at 30.0 Hz for three cycles of 20 minutes each. The chamber was opened and material adhering to the inside walls was scraped off between each cycle. White solid (84 mg, 84% yield) was recovered. X-ray powder diffraction analysis showed the solid to be the 1:1 zotepine hydrochloride benzoic acid cocrystal. Cryogrinding: Zotepine hydrochloride (717 mg, 1.95 mmol) and benzoic acid (238 mg, 1.95 mmol) were charged to a ceramic mortar. The mixture was ground using a ceramic pestle three times under nitrogen gas, and a small aliquot of the ground material was analyzed by XRPD to assess whether a cocrystal had formed. X-ray powder diffraction analysis indicated that the solids were the starting materials, and that a cocrystal had not formed.

The remaining solids were reground an additional three times for approximately 5 minutes each, with scrape-down of the walls and a half-hour rest period between grinds. A small aliquot of the grind was analyzed by XRPD to assess whether a cocrystal had formed. X-ray powder diffraction analysis again indicated that the solids were the starting materials, and that a cocrystal had not formed. The remaining solids were therefore transferred to a plastic Spex CertiPrep milling chamber (˜7.3 cm×1.9 cm) containing a stainless steel impactor (˜6.0 cm×0.9 cm) and pre-cooled in liquid nitrogen for 1 minute before milling for 5 cycles at 2.0 minutes each at 10 Hz. A 2.0-minute cooling period was conducted between each milling cycle. The grind was scraped from the walls between each cycle. A fine, white powder was recovered from the grind. X-ray powder diffraction analysis showed the solid to be the 1:1 zotepine hydrochloride benzoic acid cocrystal.

### Example 4.2

### Characterization of 1:1 Zotepine Hydrochloride Benzoic Acid Cocrystal

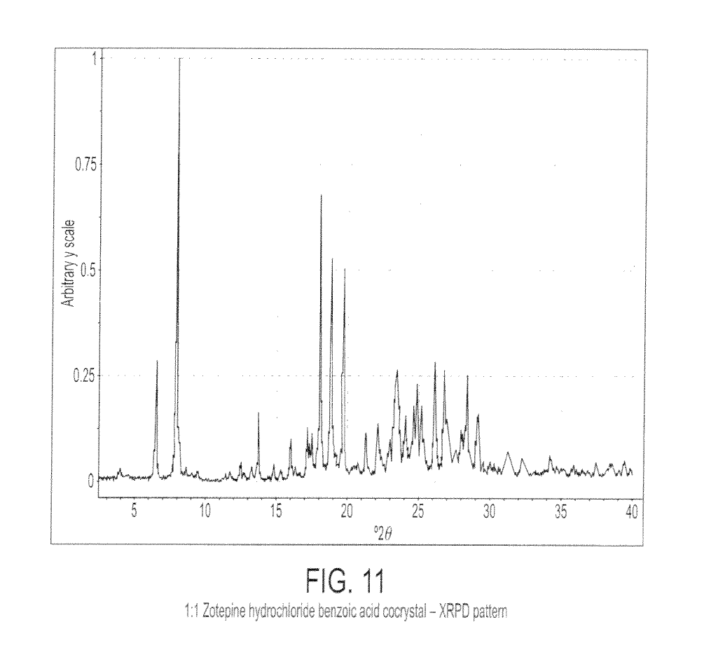

The 1:1 zotepine hydrochloride benzoic acid cocrystal was characterized by XRPD using a PANalytical X'Pert Pro diffractometer. FIG. 11 depicts the XRPD pattern of the 1:1 zotepine hydrochloride benzoic acid cocrystal. The measurement conditions are reported in Table 10. Table 11 reports the peaks identified in the XRPD pattern. The XRPD pattern, the peaks identified in the pattern or subsets of those peaks may be used to identify the 1:1 zotepine hydrochloride benzoic acid cocrystal. Peaks identified with an asterisk (*) may be considered characteristic for the 1:1 zotepine hydrochloride benzoic acid cocrystal.

TABLE 10
XRPD Measurement Conditions for 1:1 Zotepine
Hydrochloride Benzoic Acid Cocrystal

| Condition | Value |
|---|---|
| Instrument | Panalytical X-Pert Pro |
| | MPD PW3040 Pro |
| X-ray tube | Cu (1.54059 Å) |
| Voltage | 45 kV |
| Amperage | 40 mA |
| Scan range | 1.01-39.98 °2θ |
| Step size | 0.017 °2θ |
| Collection time | 1936 s |
| Scan speed | 1.2°/min |
| Slit | DS: ½°; SS: ¼° |
| Revolution time | 0.5 s |
| Mode | Transmission |

TABLE 11
Peak Positions of the XRPD Pattern for 1:1 Zotepine
Hydrochloride Benzoic Acid Cocrystal

| Degrees 2θ | Intensity % (I/Io) |
|---|---|
| 4.0 ± 0.2 | 3 |
| 6.5* ± 0.2 | 29 |
| 7.9* ± 0.2 | 100 |
| 8.6 ± 0.2 | 4 |
| 9.4 ± 0.2 | 2 |
| 11.3 ± 0.2 | 1 |
| 11.7 ± 0.2 | 2 |
| 12.4 ± 0.2 | 4 |
| 13.2 ± 0.2 | 4 |
| 13.7* ± 0.2 | 16 |
| 14.7 ± 0.2 | 4 |
| 15.2 ± 0.2 | 2 |
| 15.9 ± 0.2 | 10 |
| 16.3 ± 0.2 | 3 |
| 17.1 ± 0.2 | 13 |
| 17.2 ± 0.2 | 9 |
| 17.4 ± 0.2 | 11 |
| 18.0 ± 0.2 | 68 |
| 18.7 ± 0.2 | 52 |
| 19.6 ± 0.2 | 50 |
| 20.6 ± 0.2 | 4 |
| 21.2 ± 0.2 | 11 |
| 22.0 ± 0.2 | 13 |
| 22.3 ± 0.2 | 5 |
| 22.8 ± 0.2 | 10 |
| 23.3 ± 0.2 | 26 |
| 23.9 ± 0.2 | 15 |
| 24.5 ± 0.2 | 18 |
| 24.7 ± 0.2 | 23 |

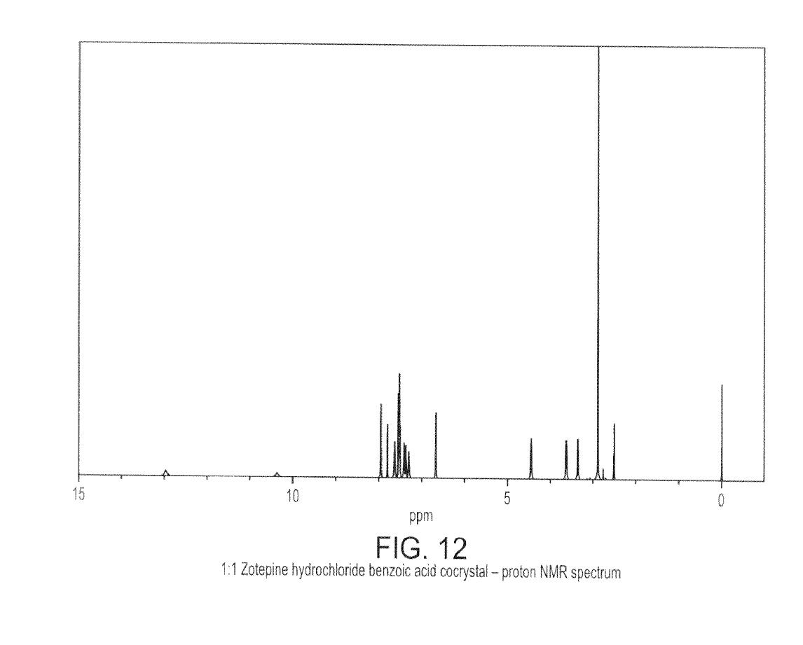

FIG. 12 depicts the proton NMR spectrum of the 1:1 zotepine hydrochloride benzoic acid cocrystal. The peaks in the solution phase ¹H NMR spectrum are reported in Table 12. Formation of the 1:1 zotepine hydrochloride benzoic acid cocrystal is confirmed by four observations. First, the chemical shifts of the CH₃, N—CH₂, O—CH₂, and NH protons were observed to be the same in the hydrochloride salt and 1:1 cocrystal, indicating the hydrochloride salt is intact in the cocrystal. Second, the appearance of a different pattern in the aromatic region (7-8 ppm) as compared to the hydrochloride salt was observed, as was an increase in the number of aromatic protons from 7 in the hydrochloride salt to 12 in the cocrystal (by integration of the aromatic region). Third, the 2 protons at 7.94-7.97 were observed at a chemical shift position expected for aromatic protons ortho to a carboxylic acid group, indicative of the presence of benzoic acid. Fourth, the appearance of the CO₂H proton of benzoic acid was observed at 12.98. That proton integrates correctly to 1.

TABLE 12
¹H NMR Peaks for 1:1 Zotepine Hydrochloride
Benzoic Acid Cocrystal

| | peak | | coupling | |
|---|---|---|---|---|
| | position | | constant | number of |
| protons | (ppm) | multiplicity | (Hz) | protons |
| CH₃ | 2.88 | singlet | — | 6 |
| CH₂N | 3.62 | triplet | 4.9 | 2 |
| CH₂O | 4.44 | triplet | 4.9 | 2 |
| C═CH | 6.66 | singlet | — | 1 |
| aromatic | 7.27-7.31 | multiplet | — | 1 |
| aromatic | 7.34-7.42 | multiplet | — | 2 |
| aromatic | 7.49-7.57 | multiplet | — | 5 |
| aromatic | 7.61-7.76 | multiplet | — | 1 |
| aromatic | 7.80 | doublet | 2.1 | 1 |
| aromatic (ortho | 7.94-7.97 | multiplet | — | 2 |
| to CO₂H) | | | | |
| NH | 10.57 | broad singlet | — | 1 |
| CO₂H | 12.98 | broad singlet | — | 1 |

FIG. 13 depicts the DSC/TGA analyses of the 1:1 zotepine hydrochloride benzoic acid cocrystal. The DSC shows a major endothermic peak with maximum at 120° C., a transition exotherm with onset at 140° C., and a secondary endotherm with peak maximum at 199° C. The TGA shows a 25% weight loss between 75 and 192° C., with decomposition after the melt.

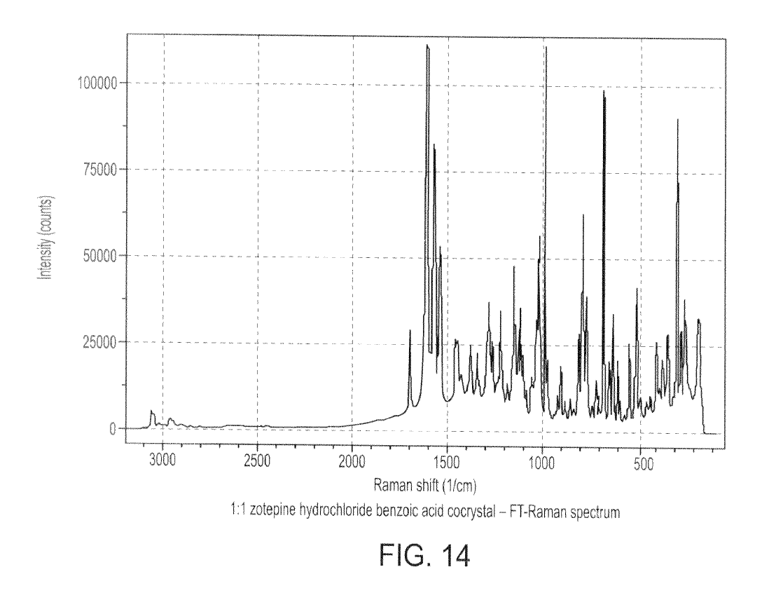

FIG. 14 depicts the FT-Raman spectrum of the 1:1 zotepine hydrochloride benzoic acid cocrystal. Table 13 reports the absorbance peaks in the Raman spectrum. The Raman data has reflections attributed to both the zotepine hydrochloride salt and benzoic acid. Slight shifting was observed when compared to the zotepine hydrochloride salt. The Raman spectrum, the peaks identified in the spectrum or subsets of those peaks may be used to identify the 1:1 zotepine hydrochloride benzoic acid cocrystal. Peaks identified with an asterisk (*) may be considered characteristic for the 1:1 zotepine hydrochloride benzoic acid cocrystal.

TABLE 13
Peaks in the Raman Spectrum of 1:1 Zotepine
Hydrochloride Benzoic Acid Cocrystal

| Peak position (cm⁻¹) | Rel. Intensity (%) |
|---|---|
| 260 ± 1 cm⁻¹ | 28 |
| 283 ± 1 cm⁻¹ | 21 |
| 304* ± 1 cm⁻¹ | 66 |
| 354 ± 1 cm⁻¹ | 20 |
| 379 ± 1 cm⁻¹ | 15 |
| 410 ± 1 cm⁻¹ | 19 |
| 445 ± 1 cm⁻¹ | 8 |
| 498 ± 1 cm⁻¹ | 8 |
| 517 ± 1 cm⁻¹ | 30 |
| 556 ± 1 cm⁻¹ | 19 |
| 605 ± 1 cm⁻¹ | 7 |
| 617 ± 1 cm⁻¹ | 15 |
| 642 ± 1 cm⁻¹ | 25 |
| 661 ± 1 cm⁻¹ | 15 |
| 692 ± 1 cm⁻¹ | 71 |
| 717 ± 1 cm⁻¹ | 8 |
| 730 ± 1 cm⁻¹ | 11 |
| 751 ± 1 cm⁻¹ | 6 |
| 783 ± 1 cm⁻¹ | 28 |
| 802* ± 1 cm⁻¹ | 46 |
| 823 ± 1 cm⁻¹ | 21 |
| 867 ± 1 cm⁻¹ | 7 |
| 895 ± 1 cm⁻¹ | 6 |
| 919 ± 1 cm⁻¹ | 14 |
| 936 ± 1 cm⁻¹ | 8 |
| 989 ± 1 cm⁻¹ | 15 |
| 1001* ± 1 cm⁻¹ | 100 |
| 1035 ± 1 cm⁻¹ | 41 |
| 1046 ± 1 cm⁻¹ | 23 |
| 1072 ± 1 cm⁻¹ | 12 |
| 1098 ± 1 cm⁻¹ | 9 |
| 1132 ± 1 cm⁻¹ | 26 |
| 1143 ± 1 cm⁻¹ | 21 |
| 1164 ± 1 cm⁻¹ | 35 |
| 1200 ± 1 cm⁻¹ | 10 |
| 1238 ± 1 cm⁻¹ | 25 |
| 1277 ± 1 cm⁻¹ | 19 |
| 1298 ± 1 cm⁻¹ | 27 |
| 1358 ± 1 cm⁻¹ | 17 |
| 1393 ± 1 cm⁻¹ | 18 |
| 1473 ± 1 cm⁻¹ | 19 |
| 1555 ± 1 cm⁻¹ | 39 |
| 1581 ± 1 cm⁻¹ | 54 |
| 1586 ± 1 cm⁻¹ | 60 |
| 1602 ± 1 cm⁻¹ | 34 |
| 1624 ± 1 cm⁻¹ | 90 |
| 1710 ± 1 cm⁻¹ | 21 |
| 2974 ± 1 cm⁻¹ | 2 |
| 3058 ± 1 cm⁻¹ | 3 |

Attempts were made to measure the aqueous equilibrium solubility at ambient temperature of the 1:1 zotepine benzoic acid cocrystal. Based on the presence of suds upon addition of solids to water, the 1:1 zotepine benzoic acid cocrystal also exhibited surface activity, but agitation of an aqueous slurry for ˜30 days followed by centrifugation afforded a hazy supernatant that was found to contain 44 mg/mL of the cocrystal by UV analysis. The solid recovered from the solubility experiment was determined to be 1:1 benzoic acid cocrystal by XRPD analysis. Thus the solubility of the 1:1 benzoic acid cocrystal is approximately 44 mg/mL.

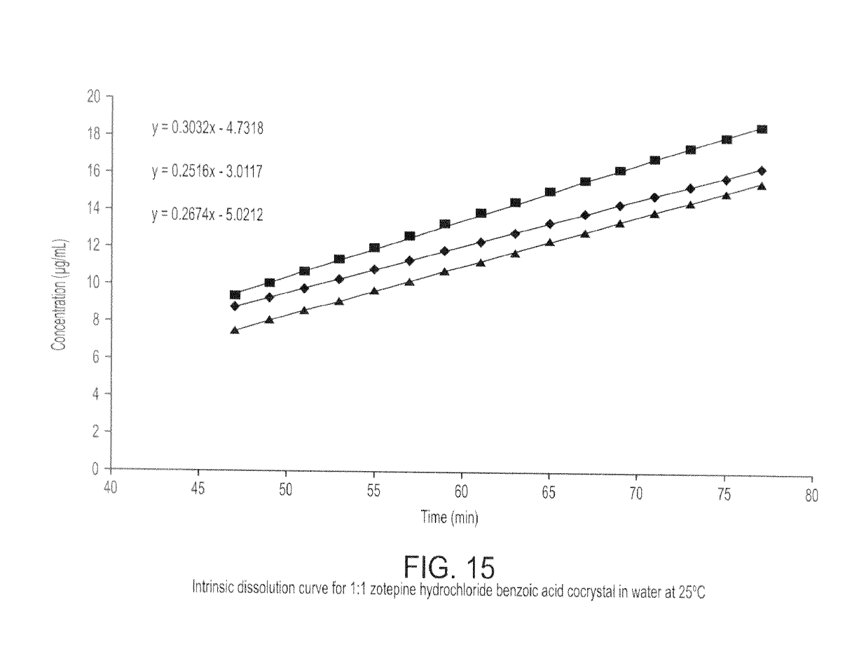

FIG. 15 depicts the intrinsic dissolution curves for the 1:1 zotepine hydrochloride benzoic acid cocrystal in water at 25° C. The intrinsic dissolution rate of the 1:1 zotepine hydrochloride benzoic acid cocrystal was 0.3 [μg/mL]/min. The solids recovered from the dissolution experiment were determined to be the 1:1 zotepine hydrochloride benzoic acid cocrystal.

### Example 4.3

### Single Crystal X-ray Structure of the 1:1 Zotepine Hydrochloride Benzoic Acid Cocrystal

Crystals of the 1:1 zotepine hydrochloride benzoic acid cocrystals were prepared at SSCI, Inc. for single crystal structure analysis by adding sufficient API to a guest-saturated ACN solution, then slurrying, as described in Example 4.1. A single crystal suitable for X-ray diffraction analysis was selected from the solids obtained. A colorless needle of the 1:1 zotepine hydrochloride benzoic acid cocrystal having approximate dimensions of 0.38×0.20×0.05 mm was selected for analysis. The structure was then determined by single crystal X-ray diffraction at the Crystallography Laboratory at Purdue University.

The crystallographic data collection and single crystal parameters for the 1:1 zotepine hydrochloride benzoic acid cocrystal are provided in Table 14.

TABLE 14
Crystal Data and Data Collection Parameters for
1:1 Zotepine Hydrochloride Benzoic Acid Cocrystal

| formula | C₂₅H₂₅Cl₂NO₃S |
|---|---|
| formula weight | 490.45 |
| space group | P c c n (No. 56) |
| a, Å | 14.1902(3) |
| b, Å | 44.5432(12) |
| c, Å | 7.5719(2) |
| V, Å³ | 4786.0(2) |
| Z | 8 |
| d_{calc}, g cm⁻³ | 1.361 |
| crystal dimensions, mm | 0.38 × 0.20 × 0.05 |
| temperature, K | 150. |
| radiation (wavelength, Å) | Mo K_{α} (0.71073) |
| monochromator | graphite |
| linear abs coef, mm⁻¹ | 0.380 |
| absorption correction applied | empirical^{a} |
| transmission factors: min, max | 0.85, 0.98 |
| diffractometer | Nonius KappaCCD |
| h, k, l range | 0 to 16 0 to 53 0 to 9 |
| 2σ range, deg | 4.65-50.07 |
| programs used | SHELXTL |
| F₀₀₀ | 2048.0 |
| weighting | |
| 1/[σ²(Fo²) + (0.0956P)² + 3.3431P] | |
| where P = (Fo² + 2Fc²)/3 | |
| data collected | 4734 |
| unique data | 4154 |
| data used in refinement | 4154 |
| cutoff used in R-factor calculations | Fₒ² > 2.0σ(Fₒ²) |
| data with I > 2.0σ(I) | 2698 |
| number of variables | 299 |
| largest shift/esd in final cycle | 0.01 |
| R(Fₒ) | 0.063 |
| R_{w}(Fₒ²) | 0.165 |
| goodness of fit | 1.061 |

^{a}Otwinowski Z. & Minor, W. Methods Enzymol. 1996, 276307.

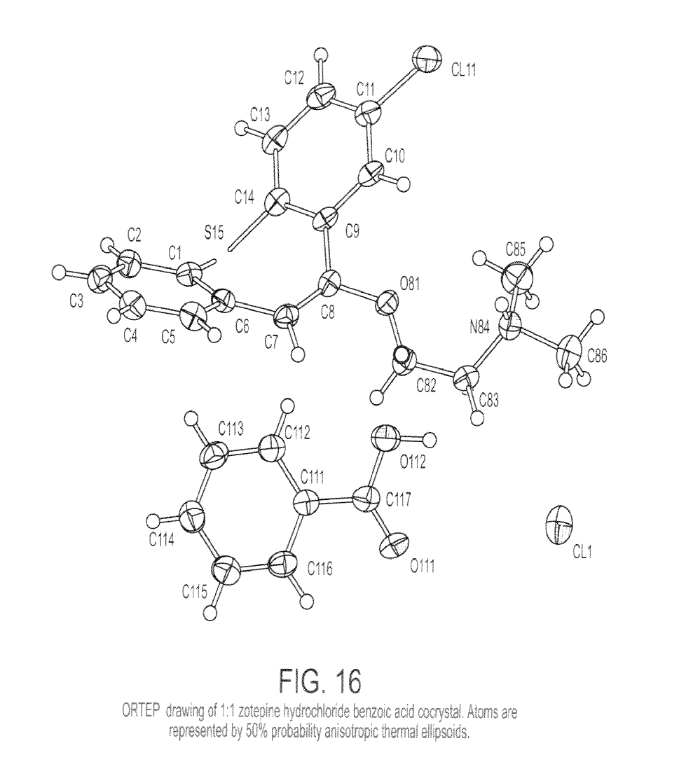

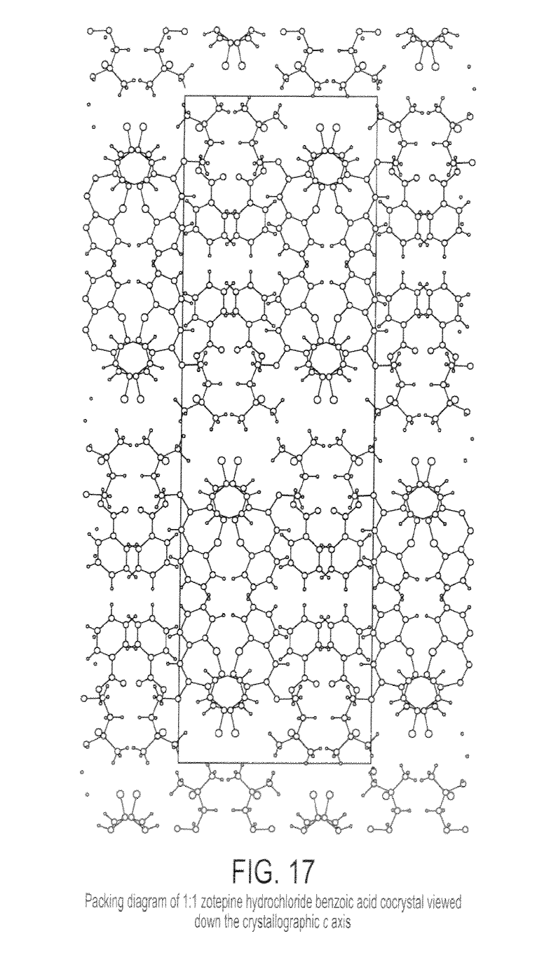

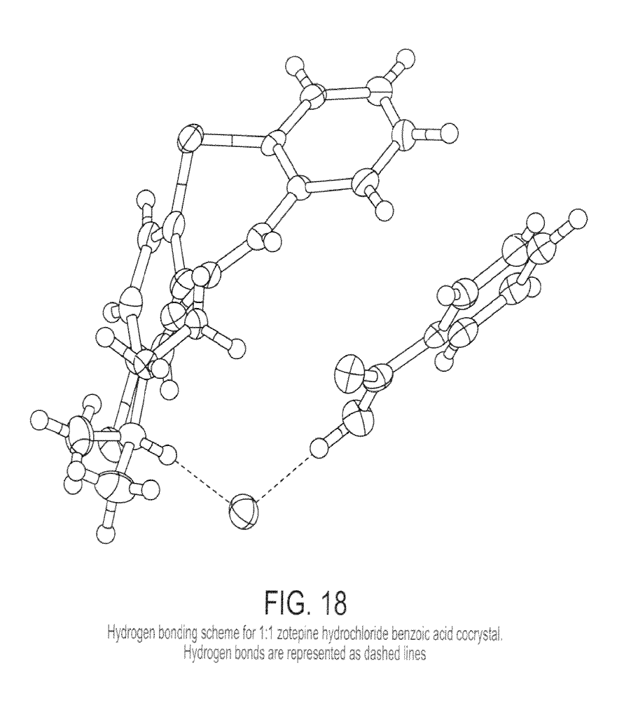

FIGS. 16-18 depict ORTEP drawings of the contents of the asymmetric unit of the 1:1 zotepine hydrochloride benzoic acid cocrystal structure. The material exhibits a layered packing motif and there are strong hydrogen bonding interactions between the chloride anion and the protons on the amine and the benzoic acid groups. FIG. 16 is an ORTEP drawing of 1:1 zotepine hydrochloride benzoic acid cocrystal. Atoms are represented by 50% probability anisotropic thermal ellipsoids. FIG. 17 is the packing diagram of 1:1 zotepine hydrochloride benzoic acid cocrystal viewed down the crystallographic c axis. FIG. 18 shows the hydrogen bonding scheme for 1:1 zotepine hydrochloride benzoic acid cocrystal. Hydrogen bonds are represented as dashed lines.

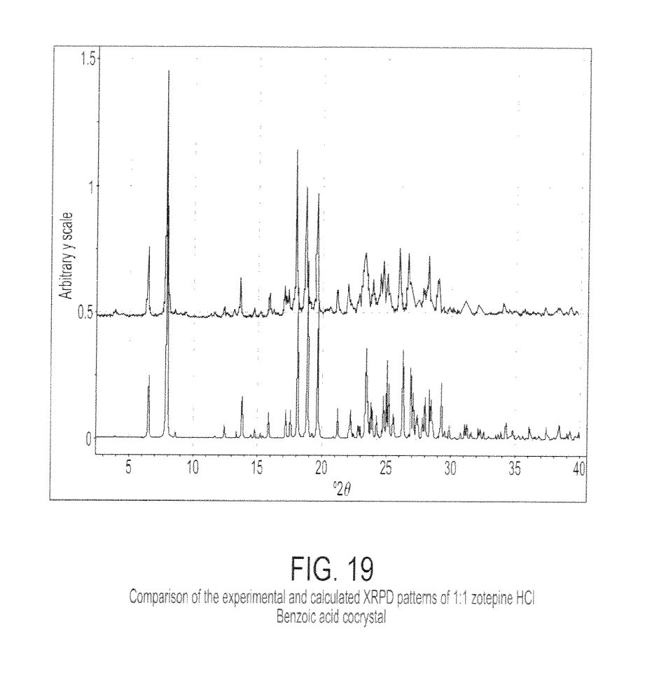

FIG. 19 compares the experimental (top) and calculated (bottom) XRPD patterns of 1:1 zotepine hydrochloride benzoic acid cocrystal. The experimental pattern was collected on a sample generated by a grinding, therefore it is possible to see additional weak reflections from the two components.

### Example 5

### Preparation and Characterization of 2:1 Zotepine Hydrochloride Benzoic Acid Cocrystal

### Example 5.1

### Preparation of 2:1 Zotepine Hydrochloride Benzoic Acid Cocrystal

Slurry Experiments

Example 5.1.1: Benzoic Acid (85 mg, 0.70 mmol), Zotepine Hydrochloride (233 mg, 0.632 mmol), and acetonitrile (1.0 mL) were added to a 2-dram vial to give white paste. The vial was placed on a pre-heated Dataplate stir plate and heated from 30 to 46° C. over approximately 2 hours with incremental addition of acetonitrile, bringing the total solvent added to 3.1 mL. Almost all of the solids were dissolved. The vial was tightly capped and placed on a rotating wheel for approximately 16 hours, during which time the mixture cooled to ambient temperature and became a thick, white paste. The paste was vacuum filtered through Whatman #1 paper, using the mother liquor to effect quantitative transfer of residual solids from the vial. The collected solid was blotted between filter paper to remove excess acetonitrile. A total of 219 mg of solid (81% yield) was recovered. Examination of the solids under a stereomicroscope revealed tiny fibrous agglomerates that were birefringent and extinguishable. X-ray powder diffraction analysis showed the solid to be the 2:1 zotepine hydrochloride benzoic acid cocrystal.

Example 5.1.2: Zotepine hydrochloride (852 mg, 2.31 mmol), benzoic acid (311 mg, 2.55 mmol), and acetonitrile (11 mL) were charged to a 20-mL scintillation vial containing a Teflon stir bar. The vial was placed on a pre-heated Dataplate stir plate and heated with stirring from 43 to 57° C. over approximately 2 hours, producing a clear solution. The stir bar was removed and the vial was placed on a rotating wheel for approximately 15 hours, during which time the mixture cooled to ambient temperature and became a thick paste. The paste was vacuum filtered through Whatman #1 paper, using the mother liquor to effect quantitative transfer of residual solids from the vial. The collected solid was dried in a vacuum oven at ambient temperature and a vacuum of approximately 30 inches mercury for approximately 6 hours. During this time, the solid was manually stirred and weighed on a balance to monitor weight loss. The sample was dried until a constant weight loss of less than 0.1% was achieved between weighings. A total of 769 mg of solid (77% yield) was recovered. X-ray powder diffraction analysis showed the solid to be the 2:1 zotepine hydrochloride benzoic acid cocrystal.

Cooling Experiment: Zotepine hydrochloride (233 mg, 0.632 mmol), benzoic acid (85 mg, 0.70 mmol), and acetonitrile (3.1 mL) were charged to a 2-dram vial. The vial was placed on a pre-heated Dataplate stir plate and heated from 40 to 50° C. over approximately 2.5 hours with magnetic stirring. An additional 0.9 mL of acetonitrile was added to produce a clear solution. Programmed cooling was conducted such that the temperature dropped from 50° C. to 36° C. over approximately 17 hours. The resulting slurry was filtered through a Magna 0.22-μm nylon membrane inside a Millipore Swinnex filter body. Mother liquor was used to effect quantitative transfer of residual solids from the vial. The filter cake was transferred to a 2-dram vial and dried in a vacuum oven at ambient temperature and a vacuum of approximately 30 inches mercury for approximately 1 hour to a constant weight of 94 mg (34% yield). X-ray powder diffraction analysis showed the solid to be the 2:1 zotepine hydrochloride benzoic acid cocrystal.

The mother liquor was returned to the original 2-dram vial and placed in a refrigerator at approximately 5° C. for 12 days. During this time, a very large, three-dimensional fibrous rosette formed. This was broken up with a microspatula, and the resulting mixture was filtered through a Magna 0.22-μm nylon membrane inside a Millipore Swinnex filter body. A fibrous mat plus aciculars were recovered, both of which were birefringent and extinguishable under a stereomicroscope. The solid was placed in a clean 2-dram vial and dried under a stream of nitrogen at ambient temperature and atmospheric pressure, but was not weighed. X-ray powder diffraction analysis showed the solid to be the 2:1 zotepine hydrochloride benzoic acid cocrystal.

The mother liquor remaining after isolation of 1:1 zotepine hydrochloride benzoic acid cocrystal (described in Example 4) spontaneously deposited crystals on standing at ambient temperature. The mixture was centrifuged for approximately 3 minutes, and the supernatant was removed by pipette. The solids were blotted between filter paper to remove excess acetonitrile. Examination of the solid under a stereomicroscope revealed a white, opaque agglomerate exhibiting birefringence. X-ray powder diffraction analysis showed the solid to be the 2:1 zotepine hydrochloride benzoic acid cocrystal.

### Example 5.2

### Characterization of 2:1 Zotepine Hydrochloride Benzoic Acid Cocrystal

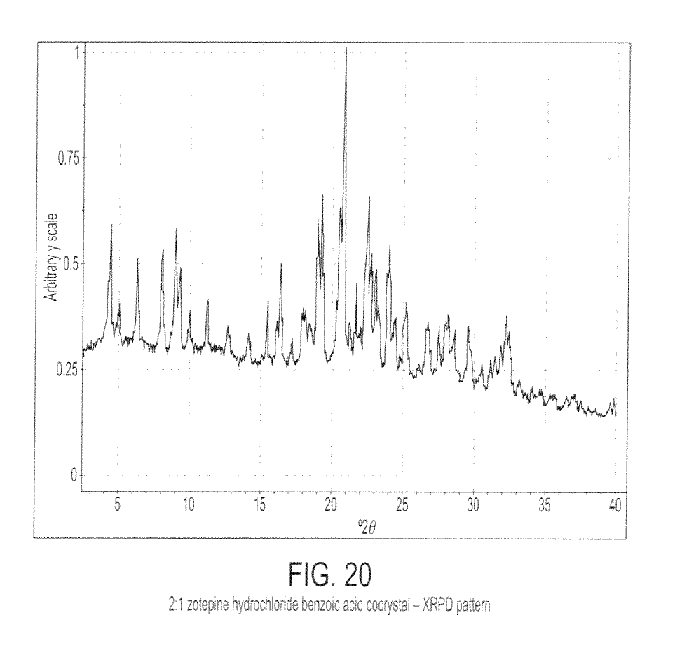

The 2:1 zotepine hydrochloride was characterized by XRPD using a Panalytical X-Pert Pro diffractometer. FIG. 20 depicts the XRPD pattern of the 2:1 zotepine hydrochloride benzoic acid cocrystal. The measurement conditions are reported in Table 15. Table 16 reports the peaks identified in the XRPD pattern. The XRPD pattern, the peaks identified in the pattern or subsets of those peaks may be used to identify the 2:1 zotepine hydrochloride benzoic acid cocrystal. Peaks identified with an asterisk (*) may be considered characteristic for the 2:1 zotepine hydrochloride benzoic acid cocrystal.

TABLE 15
XRPD Measurement Conditions for 2:1 Zotepine
Hydrochloride Benzoic Acid Cocrystal

| Condition | Value |
|---|---|
| Instrument | Panalytical X-Pert Pro |
| | MPD PW3040 Pro |
| X-ray tube | Cu (1.54059 Å) |
| Voltage | 45 kV |
| Amperage | 40 mA |
| Scan range | 1.01-39.98 °2θ |
| Step size | 0.017 °2θ |
| Collection time | 1943 s |
| Scan speed | 1.2°/min |
| Slit | DS: ½°; SS: ¼° |
| Revolution time | 0.5 s |
| Mode | Transmission |

TABLE 16
Peak Positions of the XRPD Pattern for 2:1 Zotepine
Hydrochloride Benzoic Acid Cocrystal

| Degrees 2θ | Intensity % (I/Io) |
|---|---|
| 4.4 ± 0.2 | 33 |
| 5.0* ± 0.2 | 11 |
| 6.3 ± 0.2 | 28 |
| 8.0 ± 0.2 | 34 |
| 9.0 ± 0.2 | 40 |
| 9.2 ± 0.2 | 28 |
| 9.9* ± 0.2 | 15 |
| 11.2 ± 0.2 | 17 |
| 12.6 ± 0.2 | 11 |
| 14.0 ± 0.2 | 9 |
| 15.4 ± 0.2 | 21 |
| 16.0 ± 0.2 | 14 |
| 16.3 ± 0.2 | 32 |
| 17.1 ± 0.2 | 8 |
| 17.8 ± 0.2 | 18 |
| 18.0 ± 0.2 | 17 |
| 18.4 ± 0.2 | 13 |
| 18.9 ± 0.2 | 46 |
| 19.2 ± 0.2 | 53 |
| 20.2 ± 0.2 | 19 |
| 20.5 ± 0.2 | 50 |
| 20.7 ± 0.2 | 100 |
| 21.1 ± 0.2 | 13 |
| 21.6 ± 0.2 | 26 |
| 22.3 ± 0.2 | 55 |
| 22.6 ± 0.2 | 36 |
| 23.0 ± 0.2 | 31 |
| 23.2 ± 0.2 | 20 |
| 23.8 ± 0.2 | 31 |
| 23.9 ± 0.2 | 41 |
| 24.3 ± 0.2 | 17 |
| 25.0 ± 0.2 | 24 |

FIG. 21 depicts the DSC/TGA analyses of the 2:1 zotepine hydrochloride benzoic acid cocrystal. The DSC shows a major endotherm with peak maximums at 104 and 120° C., and a minor endotherm with a peak maximum at 173° C. The TGA shows an 18% weight loss between 25 and 162° C., with weight transiently stabilizing at about 162° C. before decomposition was observed.

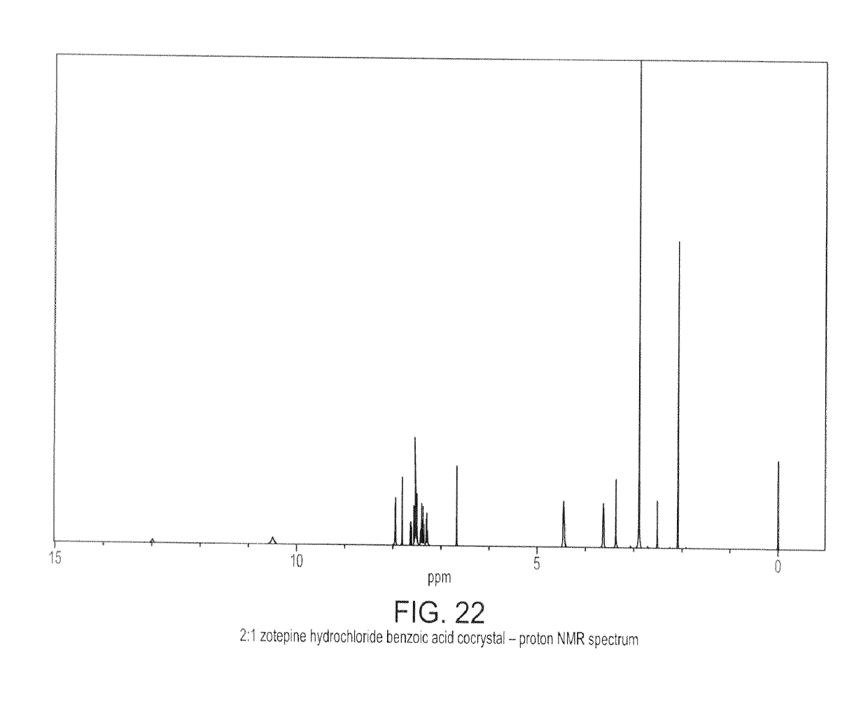

FIG. 22 depicts the proton NMR spectrum of 2:1 zotepine hydrochloride benzoic acid cocrystal. The peaks in the solution phase ¹H NMR spectrum are reported in Table 17. Formation of the 2:1 zotepine hydrochloride benzoic acid cocrystal is confirmed by four observations. First, the chemical shifts of the CH₃, N—CH₂, O—CH₂, and NH protons were observed to be the same in the hydrochloride salt and the 2:1 cocrystal, indicating the hydrochloride salt is intact in the cocrystal. Second, the appearance of a different pattern in the aromatic region (7-8 ppm) compared to the hydrochloride salt and 1:1 cocrystal was observed, as was an increase in the number of aromatic protons from 7 in the hydrochloride salt and 12 in the 1:1 cocrystal to 19 in the 2:1 cocrystal (by integration of the aromatic region). Third, the two protons at 7.94-7.98 ppm in the 1:1 cocrystal were observed to be at a chemical shift position expected for aromatic protons ortho to a carboxylic acid group, indicative of the presence of benzoic acid. The integration ratios, it is noted, indicated the presence of two such protons and two zotepine hydrochloride molecules. Fourth, the appearance of the CO₂H proton of benzoic acid was observed at 12.99 ppm. That proton integrates correctly to 1. The integration ratios, it is noted, indicated the presence of one CO₂H proton and two zotepine hydrochloride molecules.

TABLE 17
¹H NMR Peaks for 2:1 Zotepine Hydrochloride
Benzoic Acid Cocrystal

| | peak | | coupling | |
|---|---|---|---|---|
| | position | | constant | number of |
| protons | (ppm) | multiplicity | (Hz) | protons |
| CH₃ | 2.88 | singlet | — | 12 |
| CH₂N | 3.62 | triplet | 4.9 | 4 |
| CH₂O | 4.45 | triplet | 4.9 | 4 |
| C═CH | 6.66 | singlet | — | 2 |
| aromatic | 7.27-7.31 | multiplet | — | 2 |
| aromatic | 7.34-7.42 | multiplet | — | 4 |
| aromatic | 7.48-7.57 | multiplet | — | 8 |
| aromatic | 7.61-7.63 | multiplet | — | 1 |
| aromatic | 7.80 | doublet | 2.2 | 2 |
| aromatic (ortho | 7.94-7.98 | multiplet | — | 2 |
| to CO₂H) | | | | |
| NH | 10.50 | broad singlet | — | 2 |
| CO₂H | 12.99 | broad singlet | — | 1 |

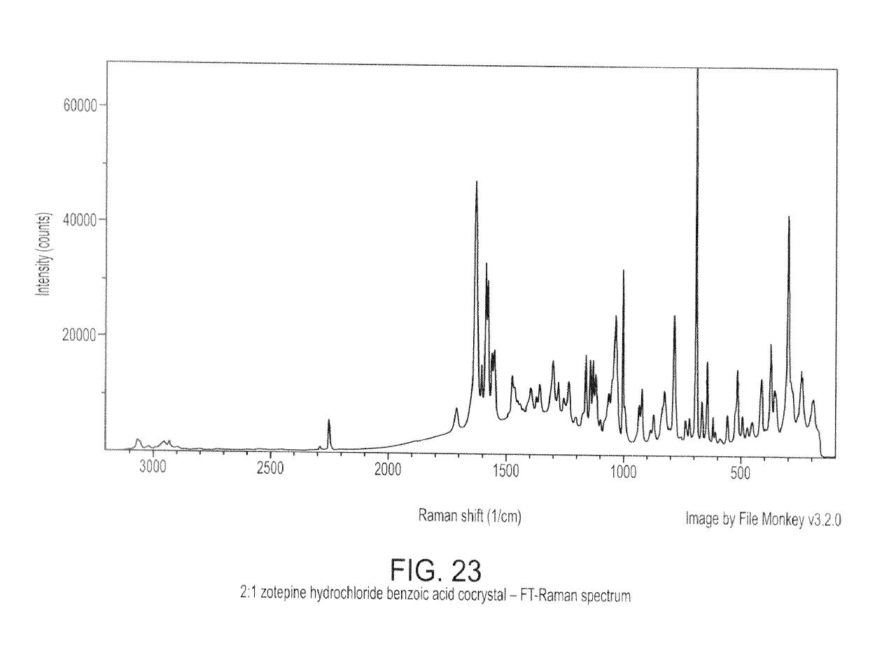

FIG. 23 depicts the FT-Raman spectrum of the 2:1 zotepine hydrochloride benzoic acid cocrystal. Table 18 reports the absorbance peaks in the Raman spectrum. The Raman data for the 2:1 cocrystal exhibits reflections attributed to both the zotepine hydrochloride salt and benzoic acid. The intensity of the reflection related to the benzoic acid is lower when compared to the 1:1 cocrystal (see Example 4.2). The Raman spectrum for the 2:1 zotepine hydrochloride benzoic acid cocrystal exhibits slight shifting when compared to the zotepine hydrochloride salt. The Raman spectrum, the peaks identified in the spectrum or subsets of those peaks may be used to identify the 2:1 zotepine hydrochloride benzoic acid cocrystal. The peaks identified with an asterisk (*) may be characteristic for the 2:1 zotepine hydrochloride benzoic acid cocrystal.

TABLE 18
Peaks in the Raman Spectrum of 2:1 Zotepine
Hydrochloride Benzoic Acid Cocrystal

| Peak position (cm⁻¹) | Rel. Intensity (%) |
|---|---|
| 193 ± 1 cm⁻¹ | 21 |
| 243 ± 1 cm⁻¹ | 28 |
| 301 ± 1 cm⁻¹ | 65 |
| 356 ± 1 cm⁻¹ | 23 |
| 374 ± 1 cm⁻¹ | 34 |
| 412 ± 1 cm⁻¹ | 26 |
| 451 ± 1 cm⁻¹ | 16 |
| 473 ± 1 cm⁻¹ | 14 |
| 492 ± 1 cm⁻¹ | 17 |
| 515 ± 1 cm⁻¹ | 28 |
| 524 ± 1 cm⁻¹ | 19 |
| 557 ± 1 cm⁻¹ | 17 |
| 586 ± 1 cm⁻¹ | 12 |
| 608 ± 1 cm⁻¹ | 13 |
| 618 ± 1 cm⁻¹ | 17 |
| 642 ± 1 cm⁻¹ | 30 |
| 665 ± 1 cm⁻¹ | 21 |
| 690 ± 1 cm⁻¹ | 100 |
| 718 ± 1 cm⁻¹ | 17 |
| 736 ± 1 cm⁻¹ | 16 |
| 783 ± 1 cm⁻¹ | 41 |
| 825 ± 1 cm⁻¹ | 23 |
| 871 ± 1 cm⁻¹ | 17 |
| 920 ± 1 cm⁻¹ | 24 |
| 933 ± 1 cm⁻¹ | 20 |
| 1004 ± 1 cm⁻¹ | 52 |
| 1034 ± 1 cm⁻¹ | 41 |
| 1062 ± 1 cm⁻¹ | 22 |
| 1097 ± 1 cm⁻¹ | 16 |
| 1118 ± 1 cm⁻¹ | 27 |
| 1130 ± 1 cm⁻¹ | 30 |
| 1141* ± 1 cm⁻¹ | 30 |
| 1160 ± 1 cm⁻¹ | 31 |
| 1201 ± 1 cm⁻¹ | 17 |
| 1234 ± 1 cm⁻¹ | 25 |
| 1255 ± 1 cm⁻¹ | 21 |
| 1277 ± 1 cm⁻¹ | 25 |
| 1300 ± 1 cm⁻¹ | 30 |
| 1357 ± 1 cm⁻¹ | 24 |
| 1371 ± 1 cm⁻¹ | 21 |
| 1395 ± 1 cm⁻¹ | 23 |
| 1474 ± 1 cm⁻¹ | 26 |
| 1549 ± 1 cm⁻¹ | 32 |
| 1560 ± 1 cm⁻¹ | 32 |
| 1577 ± 1 cm⁻¹ | 49 |
| 1586 ± 1 cm⁻¹ | 53 |
| 1604 ± 1 cm⁻¹ | 29 |
| 1630* ± 1 cm⁻¹ | 72 |
| 1710 ± 1 cm⁻¹ | 19 |
| 2252 ± 1 cm⁻¹ | 16 |
| 2932 ± 1 cm⁻¹ | 10 |
| 3062 ± 1 cm⁻¹ | 10 |

Attempts were also made to measure the aqueous equilibrium solubility at ambient temperature (25° C.) of the 2:1 zotepine hydrochloride benzoic acid cocrystal. Addition of 2:1 benzoic acid cocrystal portion-wise to water did not result in solids persisting. Complete dissolution of the 2:1 zotepine hydrochloride benzoic acid cocrystal was observed up to a concentration of 56 mg/mL, at which time the experiment was stopped, establishing a minimum value of solubility for the 2:1 zotepine hydrochloride benzoic acid cocrystal. However, a true equilibrium solubility for the 2:1 zotepine hydrochloride benzoic acid cocrystal could not be determined, as it was determined that the 2:1 cocrystal converts to the 1:1 zotepine hydrochloride benzoic acid cocrystal during dissolution experiments (XRPD analysis of solids remaining following completion of dissolution testing on the 2:1 cocrystal showed the remaining solids to be the 1:1 cocrystal). Whether the 2:1 cocrystal remained intact during the course of the experiment was not determined.

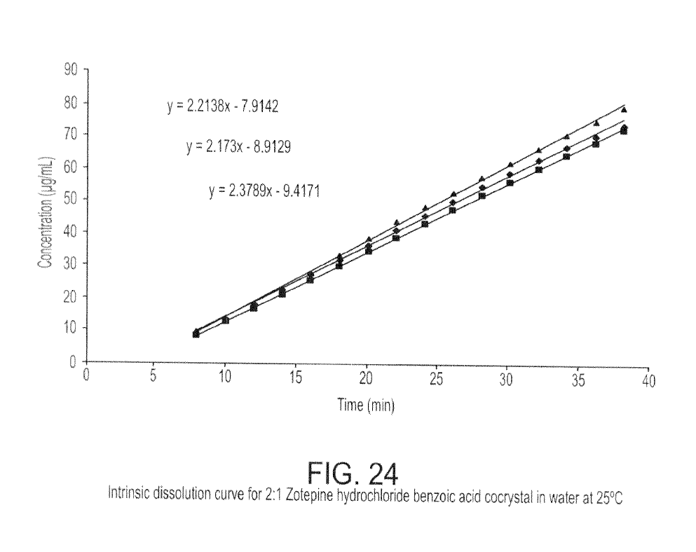

FIG. 24 depicts the intrinsic dissolution curves for the 2:1 zotepine hydrochloride benzoic acid cocrystal in water at 25° C. The intrinsic dissolution rate of the 2:1 zotepine hydrochloride benzoic acid cocrystal was 2.3 [μg/mL]/min. XRPD analysis of one of the recovered pellets of the 2:1 zotepine hydrochloride benzoic acid cocrystal following intrinsic dissolution testing showed the recovered solid to be the 1:1 zotepine hydrochloride benzoic acid cocrystal, suggesting conversion of the 2:1 zotepine hydrochloride benzoic acid cocrystal to the 1:1 benzoic acid cocrystal during the dissolution experiment. FIG. 25 shows the intrinsic dissolution comparison between the zotepine hydrochloride salt, 2:1 zotepine hydrochloride benzoic acid cocrystal, and 1:1 zotepine hydrochloride benzoic acid cocrystal in water at 25° C. (top to bottom). Concentrations for zotepine free base were not plotted, as the negligible recorded UV absorbance values for zotepine free base fell below the minimum absorbance value measured for the Beer's Law Plot relationship of absorbance vs. concentration generated from zotepine hydrochloride salt aqueous standards. If concentrations for zotepine free base were to be extrapolated from this relationship, they would fall along the x-axis of FIG. 25. The dissolution rate was highest for the zotepine hydrochloride salt (3.9 [μg/mL]/minute), followed by the 2:1 zotepine hydrochloride benzoic acid cocrystal (2.3 [μg/mL]/minute), followed by the 1:1 zotepine hydrochloride benzoic acid cocrystal (0.3 [μg/mL]/minute), and was negligible for zotepine free base.

### Comparisons of XRPD Patterns Showing Cocrystal Formation

FIG. 26 compares the XRPD patterns of crystalline zotepine free base, crystalline zotepine hydrochloride salt, 1:1 zotepine hydrochloride benzoic acid cocrystal, and benzoic acid. FIG. 27 compares the XRPD patterns of crystalline zotepine free base, crystalline zotepine hydrochloride salt, 2:1 zotepine hydrochloride benzoic acid cocrystal, and benzoic acid. FIG. 28 compares the XRPD patterns of the 1:1 zotepine hydrochloride benzoic acid cocrystal and the 2:1 zotepine hydrochloride benzoic acid cocrystal.

The unique crystal structures of the 1:1 and 2:1 cocrystals are evident by comparison of their XRPD patterns to the XRPD patterns of the components. Since each peak in an XRPD pattern represents a specific distance between atomic planes in a crystal structure, an XRPD pattern is like a fingerprint of the crystal structure of the sample analyzed. In FIG. 26 it can be seen that the numbers and angular positions (location on the x axis) of peaks in the XRPD pattern obtained from the 1:1 zotepine hydrochloride benzoic acid cocrystal differ considerably from the numbers and angular positions of peaks in the XRPD patterns obtained from zotepine hydrochloride itself and from benzoic acid itself. The presence of peaks in the XRPD pattern of the 1:1 zotepine hydrochloride benzoic acid cocrystal at positions which are devoid of peaks in the XRPD patterns of the components, such as the peaks at 6.5°2θ±0.2°2θ, 7.9°2θ±0.2°2θ, and 13.7°2θ±0.2°2θ, confirm that the cocrystal is not simply a physical mixture of the components, but exists as a unique structure. Similarly, in FIG. 27 it can be seen that the numbers and angular positions of peaks in the XRPD pattern obtained from the 2:1 zotepine hydrochloride benzoic acid cocrystal differ considerably from the numbers and angular positions of peaks in the XRPD patterns obtained from zotepine hydrochloride itself and from benzoic acid itself The presence of peaks in the XRPD pattern of the 2:1 zotepine hydrochloride benzoic acid cocrystal at positions which are devoid of peaks in the XRPD patterns of the components, such as the peaks at 5.0°2θ±0.2°2θ and 9.9°2θ±0.2°2θ, confirm that the cocrystal is not a physical mixture of the components, but exists as a unique structure.

1. Crystalline 2-[(8-chlorodibenzo[b,f]thiepin-10-yl)oxy]-N,N-dimethylethylamine hydrochloride (zotepine hydrochloride).

2. The crystalline hydrochloride salt of claim 1, characterized by a powder x-ray diffraction pattern having peaks at 9.4°2θ±0.2°2θ, 11.7°2θ±0.2°2θ, and 12.7°2θ±0.2°2θ.

3. The crystalline hydrochloride salt of claim 2, further characterized by a Raman spectrum having peaks at 645 cm⁻¹±1 cm⁻¹, 788 cm⁻¹±1 cm⁻¹, and 1032 cm⁻¹±1 cm⁻¹.

4. A pharmaceutical composition for treating a central nervous system disorder, comprising a therapeutically effective amount of the crystalline hydrochloride salt of claim 1 and a pharmaceutically acceptable carrier.

5. A method for treating a central nervous system disorder in a mammal, comprising administering to a patient in need thereof a therapeutically effective amount of the crystalline hydrochloride salt of claim 1.

6. The method of claim 5, wherein the central nervous system disorder is selected from the group consisting of schizophrenia, psychosis, cognitive symptoms of schizophrenia or psychosis, negative symptoms of schizophrenia or psychosis, bipolar disorder, Huntington's Disease, behavioral and psychological symptoms of dementia, pain, gout, depression, and anxiety disorders.

7. The method of claim 6, wherein the central nervous system disorder is schizophrenia, psychosis, or bipolar disorder.

8. A 1:1 2-[(8-chlorodibenzo[b,f]thiepin-10-yl)oxy]-N,N-dimethylethylamine hydrochloride (zotepine hydrochloride) benzoic acid cocrystal.

9. The cocrystal of claim 8, characterized by a powder x-ray diffraction pattern having peaks at 6.5°2θ±0.2°2θ, 7.9°2θ±0.2°2θ, and 13.7°2θ±0.2°2θ.

10. The cocrystal of claim 9, further characterized by a Raman spectrum having peaks at 304 cm⁻±1 cm⁻¹, 802 cm⁻¹±1 cm⁻¹, and 1001 cm⁻¹.

11. A pharmaceutical composition for treating a central nervous system disorder, comprising a therapeutically effective amount of a cocrystal of claim 8 and a pharmaceutically acceptable carrier.

12. A method for treating a central nervous system disorder in a mammal, comprising administering to a patient in need thereof a therapeutically effective amount of a cocrystal of claim 8.

13. The method of claim 12, wherein the central nervous system disorder is selected from the group consisting of schizophrenia, psychosis, cognitive symptoms of schizophrenia or psychosis, negative symptoms of schizophrenia or psychosis, bipolar disorder, Huntington's Disease, behavioral and psychological symptoms of dementia, pain, gout, depression, and anxiety disorders.

14. The method of claim 13, wherein the central nervous system disorder is schizophrenia, psychosis, or bipolar disorder.

15. A 2:1 2-[(8-chlorodibenzo[b,f]thiepin-10-yl)oxy]-N,N-dimethylethylamine hydrochloride (zotepine hydrochloride) benzoic acid cocrystal.

16. The cocrystal of claim 15, characterized by a powder x-ray diffraction pattern having peaks at 5.0°2θ±0.2°2θ and 9.9°2θ±0.2°2θ.

17. The cocrystal of claim 16, further characterized by a Raman spectrum having peaks at 1004 cm⁻¹±1 cm⁻¹, 1141 cm⁻¹±1 cm⁻¹, and 1630 cm⁻¹±1 cm⁻¹.

18. A pharmaceutical composition for treating a central nervous system disorder, comprising a therapeutically effective amount of a cocrystal of claim 15 and a pharmaceutically acceptable carrier.

19. A method for treating a central nervous system disorder in a mammal, comprising administering to a patient in need thereof a therapeutically effective amount of a cocrystal of claim 15.

20. The method of claim 19, wherein the central nervous system disorder is selected from the group consisting of schizophrenia, psychosis, cognitive symptoms of schizophrenia or psychosis, negative symptoms of schizophrenia or psychosis, bipolar disorder, Huntington's Disease, behavioral and psychological symptoms of dementia, pain, gout, depression, and anxiety disorders.

21. The method of claim 20, wherein the central nervous system disorder is schizophrenia, psychosis, or bipolar disorder.
